(19) United States
(12) Patent Application Publication
(10) Pub. No.: US 2018/0278609 A1
(43) Pub. Date: Sep. 27, 2018

(54) INFORMATION PROCESSING APPARATUS AND INFORMATION PROCESSING METHOD

(71) Applicant: FUJITSU LIMITED, Kawasaki-shi (JP)

(72) Inventors: Hiroyuki ANDO, Sayama (JP); Akito YAMAZAKI, Kawasaki (JP); Kazunori Kobashi, Yamato (JP)

(73) Assignee: FUJITSU LIMITED, Kawasaki-shi (JP)

(21) Appl. No.: 15/878,473

(22) Filed: Jan. 24, 2018

(30) Foreign Application Priority Data
Mar. 27, 2017 (JP) 2017-060363

(51) Int. Cl. H04L 29/06; H04L 29/08; G06F 17/30; A61B 5/00; G16H 10/60; G06Q 50/22

(52) U.S. Cl. CPC H04L 63/0884; H04L 67/28; G06Q 50/22; A61B 5/00; G16H 10/60; G06F 17/30595

(57) ABSTRACT

An information processing apparatus includes a memory and a processor coupled to the memory and configured to: store personal identification information indicating a person and proxy identification information indicating a proxy who acts on behalf of the person by associating the personal identification information and the proxy identification information with each other; obtain the personal identification information from the memory, which stores the personal identification information and personal information of the person in association with each other, based on the proxy identification information presented by the proxy; obtain the personal information from the memory based on the personal identification information obtained from the memory; and output the personal information obtained.

### CROSS-REFERENCE TO RELATED APPLICATION

This application is based upon and claims the benefit of priority of the prior Japanese Patent Application No. 2017-060363, filed on Mar. 27, 2017, the entire contents of which are incorporated herein by reference.

### FIELD

The embodiments discussed herein are related to an information processing apparatus, a computer-readable recording medium, and an information processing method.

### BACKGROUND

Recently, the necessity of an analysis of big data is gradually increased. In the analysis of big data, in order to obtain a more accurate and useful analysis result, it is desirable to collect data samples as many as possible.

There may be a plan on a government level, for example, to implement a future policy that promotes an analysis of big data in a domestic medical field. According to the policy, it is expected that data of an electronic medical record of a hospital is collected, the collected data is processed as anonymous data, and the anonymous data may be provided to a group desiring to use/utilize data as the data usable for an analysis of big data.

There is a known technology for using medical record information of a patient or the like.

Related technologies are disclosed in, for example, Japanese Laid-Open Patent Publication Nos. 2016-218644, 2002-117142, 2001-318992, 2007-188290, 2002-288341, and 2012-212199.

### SUMMARY

According to an aspect of the embodiments, an information processing apparatus is provided including a memory and a processor coupled to the memory and configured to store personal identification information indicating a person and proxy identification information indicating a proxy who acts on behalf of the person by associating the personal identification information and the proxy identification information with each other; obtain the personal identification information from the memory, which stores the personal identification information and personal information of the person in association with each other, based on the proxy identification information presented by the proxy; obtain the personal information from the memory based on the personal identification information obtained from the memory; and output the personal information obtained.

The object and advantages of the invention will be realized and attained by means of the elements and combinations particularly pointed out in the claims. It is to be understood that both the foregoing general description and the following detailed description are exemplary and explanatory and are not restrictive of the invention, as claimed.

### BRIEF DESCRIPTION OF DRAWINGS

FIG. 1 is a functional configuration diagram of an information processing apparatus;

FIG. 2 is a flowchart of information processing;

FIG. 3 is a configuration diagram of an information processing system;

FIG. 4 is a diagram illustrating patient information and proxy information;

FIG. 5 is a diagram illustrating a table storing basic patient information;

FIG. 6 is a diagram illustrating a table storing basic medical information;

FIG. 7 is a diagram illustrating a table storing information about an examining medical institution;

FIG. 8 is a diagram illustrating a table storing information about a proxy and a client;

FIG. 9 is a diagram illustrating a table storing identity authentication information;

FIG. 10A is a diagram (1) illustrating medical institution information;

FIG. 10B is a diagram (2) illustrating medical institution information;

FIG. 11 is a diagram illustrating a table storing unique information about a medical institution;

FIG. 12 is a diagram illustrating a table storing information about an affiliated doctor and the like;

FIG. 13 is a diagram illustrating a table storing information about an affiliated office employee and the like;

FIG. 14 is a diagram illustrating a table storing information about a patient;

FIG. 15 is a diagram illustrating a table storing information about institution authentication;

FIG. 16A is a diagram (1) illustrating an ID obtaining sequence;

FIG. 16B is a diagram (2) illustrating an ID obtaining sequence;

FIG. 17A is a diagram (1) illustrating a medical examination sequence;

FIG. 17B is a diagram (2) illustrating a medical examination sequence;

FIG. 17C is a diagram (3) illustrating a medical examination sequence;

FIG. 17D is a diagram (4) illustrating a medical examination sequence;

FIG. 17E is a diagram (5) illustrating a medical examination sequence;

FIG. 17F is a diagram (6) illustrating a medical examination sequence;

FIG. 18A is a diagram (1) illustrating an ID obtaining sequence of registering a proxy;

FIG. 18B is a diagram (2) illustrating an ID obtaining sequence of registering a proxy;

FIG. 19A is a diagram (1) illustrating proxy registration processing;

FIG. 19B is a diagram (2) illustrating proxy registration processing;

FIG. 20A is a diagram (1) illustrating proxy check processing;

FIG. 20B is a diagram (2) illustrating proxy check processing;

FIG. 20C is a diagram (3) illustrating proxy check processing;

FIG. 21A is a diagram (1) illustrating a medical examination sequence based on identity confirmation of a proxy;

FIG. 21B is a diagram (2) illustrating a medical examination sequence based on identity confirmation of a proxy;

FIG. 21C is a diagram (3) illustrating a medical examination sequence based on identity confirmation of a proxy;

FIG. 21D is a diagram (4) illustrating a medical examination sequence based on identity confirmation of a proxy;

FIG. 21E is a diagram (5) illustrating a medical examination sequence based on identity confirmation of a proxy;

FIG. 21F is a diagram (6) illustrating a medical examination sequence based on identity confirmation of a proxy;

FIG. 21G is a diagram (7) illustrating a medical examination sequence based on identity confirmation of a proxy;

FIG. 21H is a diagram (8) illustrating a medical examination sequence based on identity confirmation of a proxy; and

FIG. 22 is a configuration diagram of hardware of an information processing apparatus.

### DESCRIPTION OF EMBODIMENTS

In a medical institution such as a hospital, it may be difficult to confirm the identity of a patient when past medical examination information of the patient and the like is re-used from the electronic medical record collected based on the foregoing policy of the government.

Such problem is not limited to the case where the medical examination information of the electronic medical record is re-used in the medical institution, and also arises in the case where other personal information is used in other information using institutions.

Hereinafter, an exemplary embodiment will be described in detail with reference to the drawings.

Electronic medical record is data including a lot of elements of personal information related to patient's privacy, but for medical institutions such as hospitals, it is also greatly useful information when examining a patent. Accordingly, it is desirable for a medical institution to be able to re-use past medical examination information of a patient from the collected electronic medical record on the premise of permission of the patient. An example of the past medical examination information may include medical examination information when a patient is examined in another medical institution.

However, when a patient falls into a critical condition of unconsciousness due to a traffic accident or the like, the patient's identity is not known in the medical institution because the patient is in an unconsciousness state. Further, a patient's identity is still unknown when a patient is old and there is no proper response to a medical examination inquiry. As described above, when it is difficult to confirm an identity of a patient, it is difficult to obtain past medical examination information.

Even though a medical institution manages to know the identity of a patient in some way, it is difficult to confirm a result of a medical examination by the patient, and it is difficult to confirm whether the patient permits a re-use of the medical examination information because the patient is unconscious and the intention of the patent is unknown.

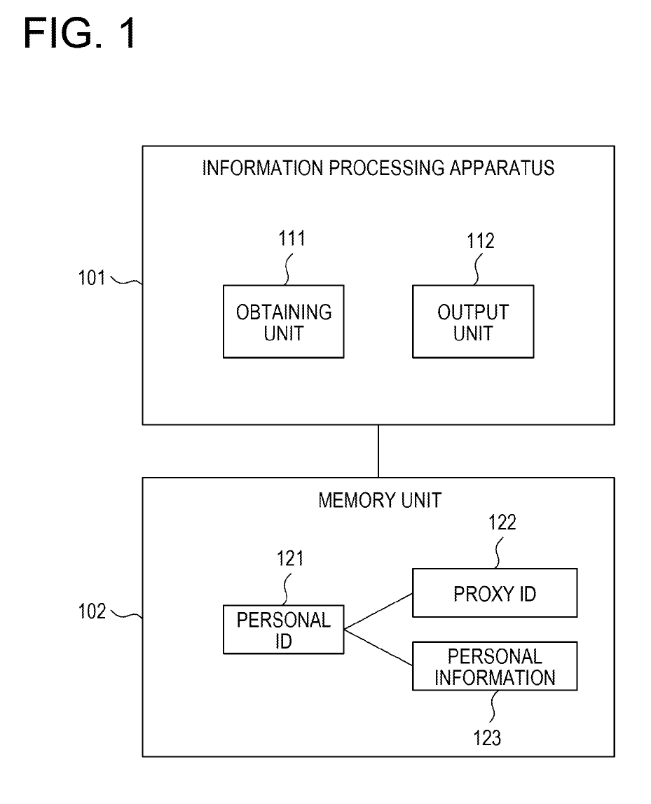

FIG. 1 illustrates a functional configuration example of an information processing apparatus (computer) according to an exemplary embodiment. An information processing apparatus 101 of FIG. 1 includes an obtaining unit 111 and an output unit 112. A memory unit 102 stores personal identification information (personal ID) 121 indicating a person and proxy identification information (proxy ID) 122 indicating a proxy who acts on behalf of the person in association with each other, and stores the personal ID 121 and personal information 123 of the person in association with each other. The information processing apparatus 101 may include the memory unit 102.

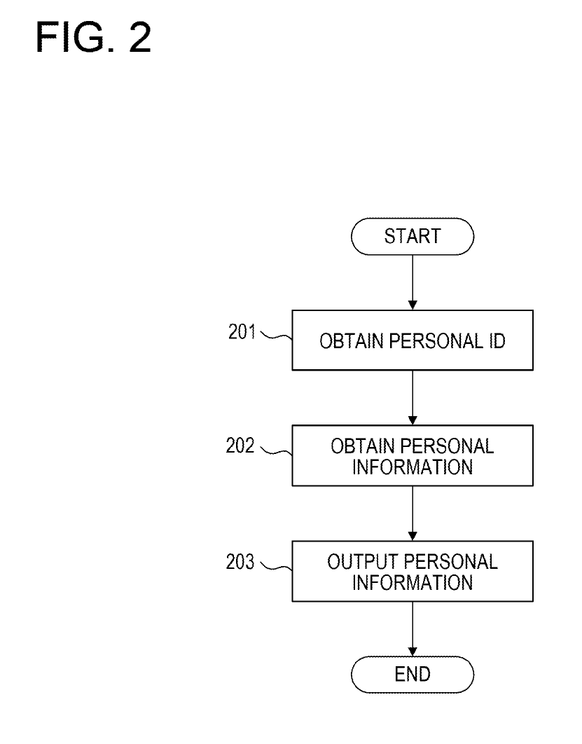

FIG. 2 is a flowchart illustrating an example of information processing performed by the information processing apparatus 101 of FIG. 1. First, the obtaining unit 111 obtains a personal ID 121 based on a proxy ID presented by a proxy from the memory unit 102 (step 201), and obtains personal information 123 from the memory unit 102 based on the obtained personal ID 121 (step 202). Further, the output unit 112 outputs the personal information 123 obtained by the obtaining unit 111 (step 203).

According to the information processing apparatus 101, even when it is difficult to confirm an identity of a person, personal information of the person may be used.

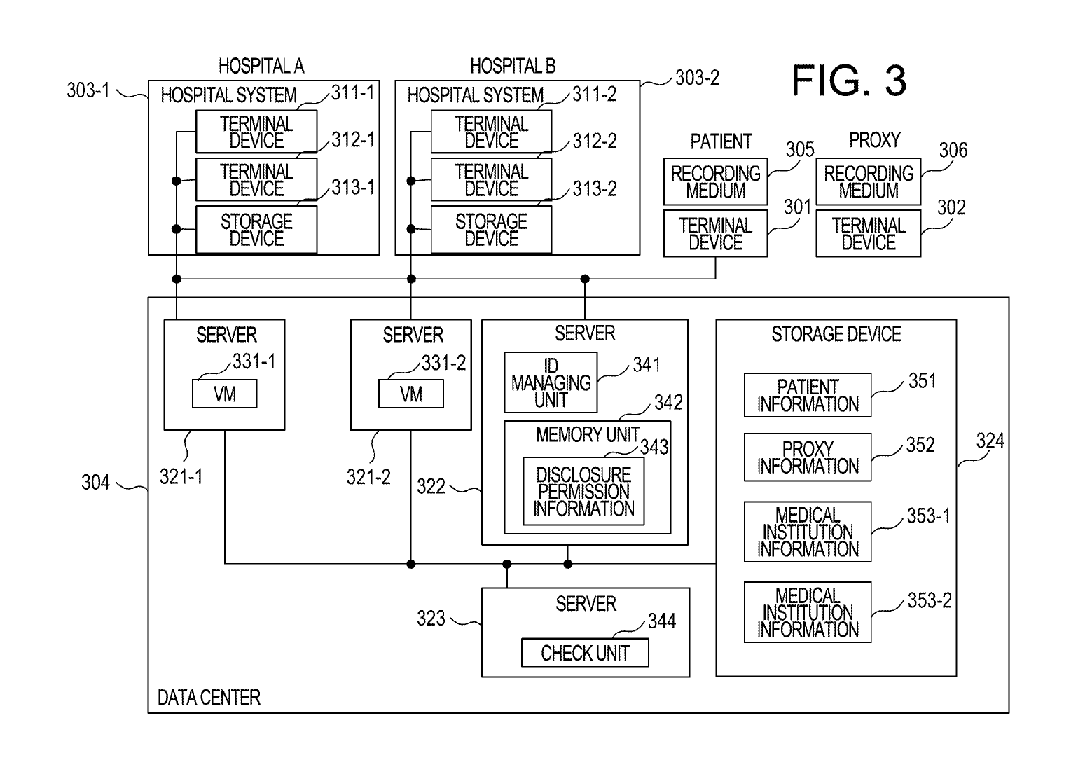

FIG. 3 illustrates a configuration example of an information processing system including the information processing apparatus 101 of FIG. 1. The information processing system of FIG. 3 is an electronic medical record system collecting electronic medical record, and includes a terminal device 301 of a patient, a terminal device 302 of a proxy who acts on behalf of a patient, a hospital system 303-1 of hospital A, a hospital system 303-2 of hospital B, and a data center 304. The data center 304 is used by an information collecting institution which collects and manages an electronic medical record.

The hospital A and the hospital B are information providing institutions which provide an information collecting institution with data of the electronic medical record. The hospital A and the hospital B are also information using institutions which use data of the collected electronic medical records. The number of hospital systems is not limited to two, and when the number of hospitals is three or more, the number of hospital systems is three or more. The hospitals may be, for example, hospitals distributed throughout the entire nation and may be hospitals located in a specific region.

The hospital system 303-3 (i=1 and 2) includes a terminal device 311-i of a doctor, a terminal device 312-i of an office employee, and a storage device 313-i. The storage device 313-i is a local storage which stores an electronic medical record including medical examination information about a patient.

The data center 304 includes a server 321-1, a server 321-2, a server 322, a server 323, and a storage device 324. Within the server 321-1, a virtual machine (VM) 331-1 of the hospital A is operated, and within the server 321-2, a VM 331-2 of the hospital B is operated. The server 322 includes an ID managing unit 341 and a memory unit 342, and the memory unit 342 stores disclosure permission information 343. The server 323 includes a check unit 344.

The storage device 324 stores patient information 351, proxy information 352, medical institution information 353-1 of the hospital A, and medical institution information 353-2 of the hospital B. The patient information 351 is information about a patient, and the proxy information 352 is information about a proxy. The medical institution information 353-1 is information about the hospital A, and the medical institution information 353-2 is information about the hospital B.

The server 321-i may communicate with the terminal device 311-i of the hospital system 303-i, a terminal device 312-i, and the storage device 313-i via a communication network. The server 322 may communicate with the terminal device 301, the terminal device 311-i, and the terminal device 312-i via the communication network. Further, the server 321-i, the server 322, the server 323, and the storage device 324 may communicate with one another via a communication network within the data center 304.

The VM 331-i receives information of the electronic medical record transmitted from the hospital system 303-i and transmits the received information to the ID managing unit 341. The ID managing unit 341 writes the information of the electronic medical record in the patient information 351 and the medical institution information 353-i. In this case, the medical examination information of the patient included in the electronic medical record is recorded in the medial institution information 353-i. Further, the ID managing unit 341 sets in the disclosure permission information 343 whether to permit the disclosure of the medical institution information 353-i to another hospital system 303-j that is different from the hospital system 303-i.

A determination whether to permit the disclosure of the medical institution information 353-i may be made by the patient. However, when it is difficult to confirm an intention of the patient, a proxy may also permit the disclosure of the medical institution information 353-i on behalf of the patient. The check unit 344 checks eligibility of the proxy and excludes a registration of an ineligible proxy and also excludes a permission of the disclosure of information by the ineligible proxy.

A recording medium 305 of a patient and a recording medium 306 of a proxy are computer readable recording media. The recording medium 305 stores a medical ID given to a patient, and the recording medium 306 stores a medical ID given to a proxy.

The server 322 corresponds to the information processing apparatus 101 of FIG. 1, the ID managing unit 341 corresponds to the obtaining unit 111, and the storage device 324 corresponds to the memory unit 102. In this case, a communication interface (not illustrated) included in the server 322 corresponds to the output unit 112. The medical ID of the patient stored in the recording medium 305 corresponds to the personal ID 121 and the medical ID of the proxy stored in the recording medium 306 corresponds to the proxy ID 122. The patient information 351 and the medical examination information recorded in the medical institution information 353-i correspond to the personal information 123.

When the information recorded in the patient information 351, the proxy information 352, and the medical institution information 353-i is updated, the ID managing unit 341 stops a use of the information without deleting the information before the update, and additionally writes the information after the update. Accordingly, the information before the update is recorded as a history.

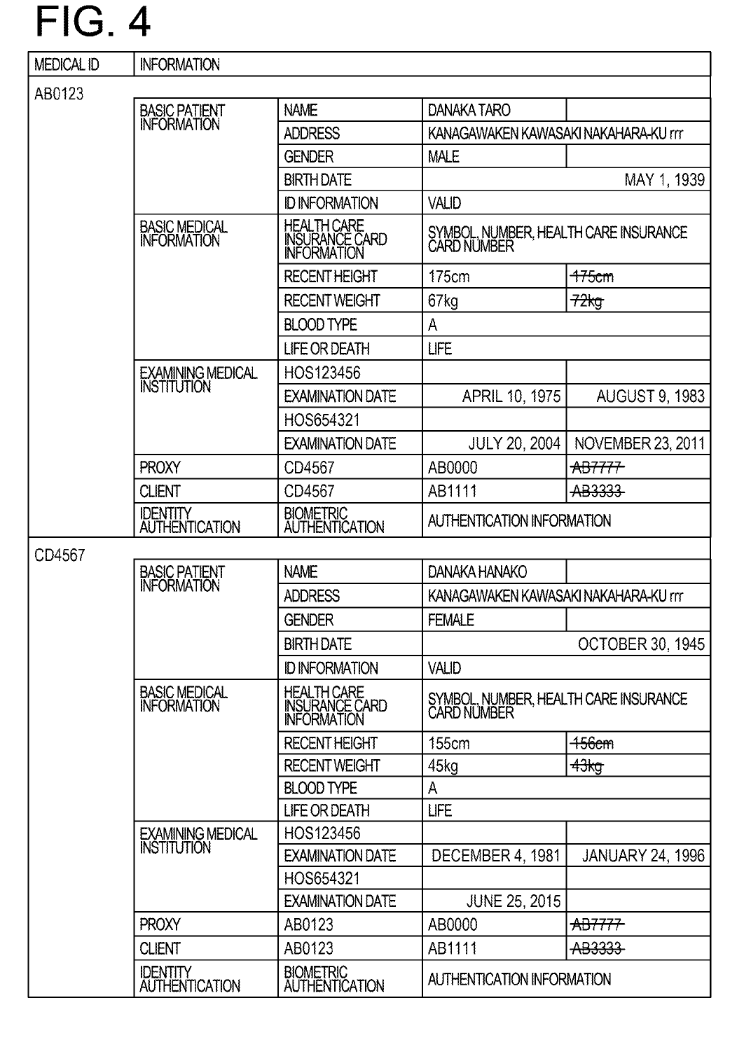

FIG. 4 illustrates an example of the patient information 351 and the proxy information 352. The information of FIG. 4 includes information about a patient indicated by a medical ID “AB0123” and information about a patient indicated by a medical ID “CD4567”. The information about each patient includes items of basic patient information, basic medical information, an examining medical institution, a proxy, a client, and identity authentication.

The basic patient information is basic information about the patient, and includes items of a name, an address, gender, a birth date, and ID information. The ID information indicates whether a medical ID of the patient is effective. The basic medical information includes items of health care insurance card information, recent height, recent weight, a blood type, and life or death. A numerical value cancelled with a strike-out line among the numerical values recorded in the recent height item indicates a past measurement value, and a numerical value having no strike-out line indicates a recent measurement value. The same is applied to the numerical values recorded in the recent weight item.

The examining medical institution includes an item of a medical examination date for each medical institution ID. In the present example, a medical examination date of the hospital A indicated by the medical institution ID “HOS123456” and a medical examination date of the hospital B indicated by the medical institution ID “HOS654321” are recorded.

A medical ID of a proxy who acts on behalf of a patient is recorded in the item of the proxy, and a medical ID of another patient who the patient himself/herself acts on behalf of is recorded in the item of the client. As described above, in the information of FIG. 4, a medical ID of a patient who is a client and a medical ID of a proxy who acts on behalf of the patient are recorded in association with each other. Among the medical IDs recorded in the item of the proxy, a medical ID cancelled with a strike-out line indicates a past proxy, and a medical ID having no strike-out line indicates a present proxy. The same is applied to a medical ID recorded in the item of the client.

In the present example, two medical IDs including “CD4567” and “AB0000” are recorded as the proxies of the patient indicated by the medical ID “AB0123”, and two medical IDs including “CD4567” and “AB1111” are recorded as the clients of the same patient. Accordingly, the patient indicated by the medical ID “CD4567” is the proxy representing the patient indicated by the medical ID “AB0123” and is also the client. When it is assumed that information about the patient indicated by the medical ID “AB0123” is patient information 351, information about the patient indicated by the medical ID “CD4567” corresponds to proxy information 352.

Similarly, two medical IDs including “AB0123” and “AB0000” are recorded as the proxies of the patient indicated by the medical ID “CD4567” and two medical IDs including “AB0123” and “AB1111” are recorded as the clients of the same patient. Accordingly, the patient indicated by the medical ID “AB0123” is the proxy representing the patient indicated by the medical ID “CD4567” and is also the client. When it is assumed that information about the patient indicated by the medical ID “CD4567” is patient information 351, information about the patient indicated by the medical ID “AB0123” corresponds to proxy information 352.

The proxy relation may be incurred in some cases such as, for example, when the two patients illustrated in FIG. 4 are a husband and a wife or a parent and a child. The proxy relation between the information of the proxy and the client is recorded and consistency between the information of the proxy and the client is maintained, so that the number of patient who a specific proxy acts on behalf of may be recognized.

An authentication method and authentication information of a patient are recorded in the item of the identity authentication. For example, when an authentication method is biometric authentication, feature data indicating biometric feature, such as a fingerprint, a palm pattern, a vein pattern, a face image, and a voice print, is recorded as authentication information.

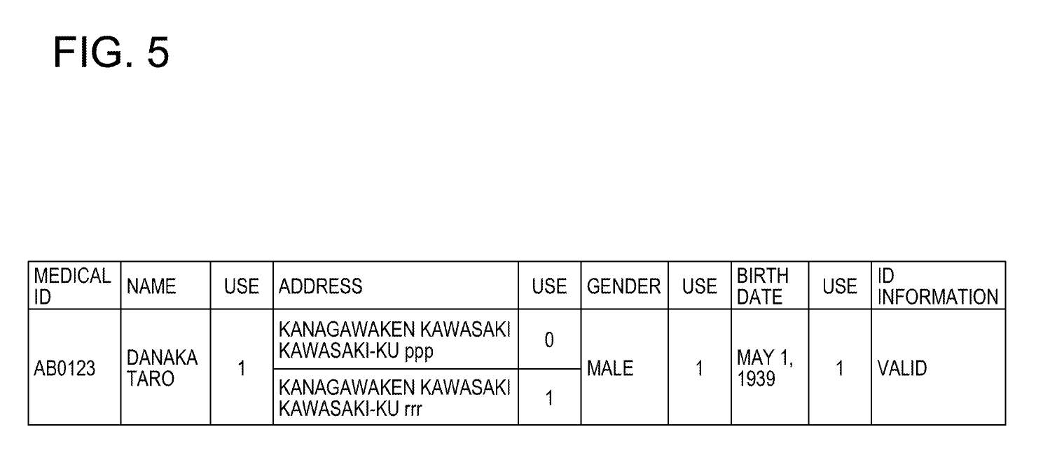

FIG. 5 illustrates an example of a table storing basic patient information about a patient indicated by the medical ID “AB0123” of FIG. 4. The table of FIG. 5 includes items of a medical ID, a name, an address, gender, a birth date, and ID information of a patient.

A flag of “use” provided at a right side of the respective items indicates whether the information is used. A flag value “1” indicates that the information is used, and a flag value “0” indicates that the information is not used. For example, when the information is updated, “0” is set to the information before update, and “1” is set to the information after update. Further, when the information is deleted, “0” is set in the deleted information.

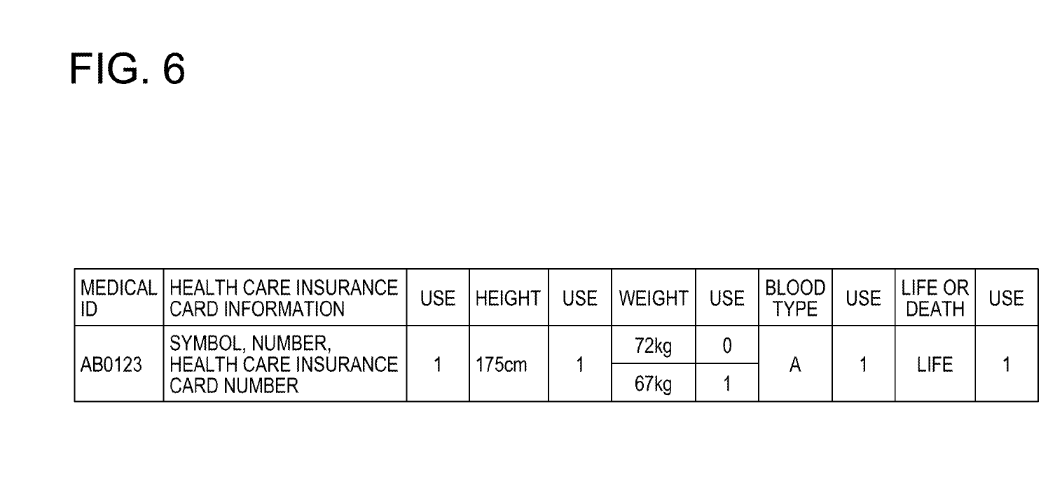

FIG. 6 illustrates an example of a table storing basic medical information about a patient indicated by the medical ID “AB0123” of FIG. 4. The table of FIG. 6 includes items of a medical ID, health care insurance card information, height, weight, a blood type, and life or death of a patient. A description of information indicated by a flag of “use” is the same as the description of FIG. 5.

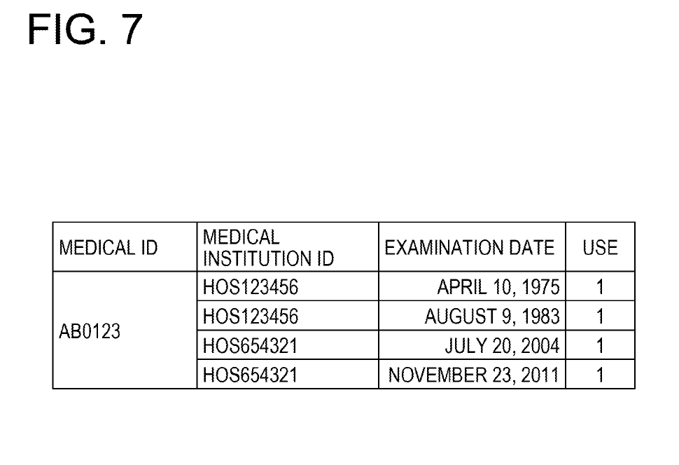

FIG. 7 illustrates an example of a table storing information about a medical institution examining a patient indicated by the medical ID “AB01234” of FIG. 4. The table of FIG. 7 includes items of a medical ID of a patient, a medical institution ID, and a medical examination date. A description of information indicated by a flag of “use” is the same as the description of FIG. 5.

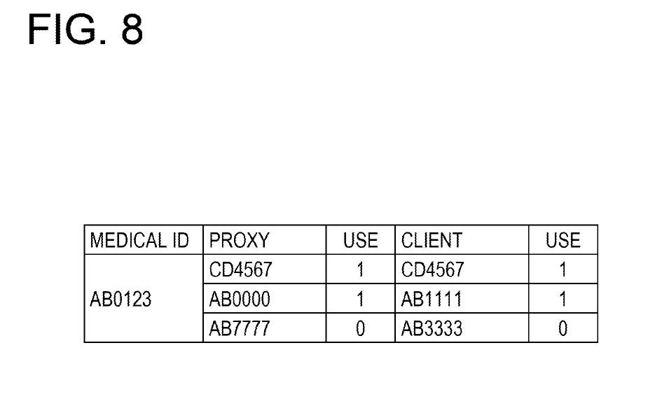

FIG. 8 illustrates an example of a table storing information about a proxy and a client of a patient indicated by the medical ID “AB0123” of FIG. 4. The table of FIG. 8 includes items of a medical ID of a patient, a proxy, and a client. A description of information indicated by a flag of “use” is the same as the description of FIG. 5.

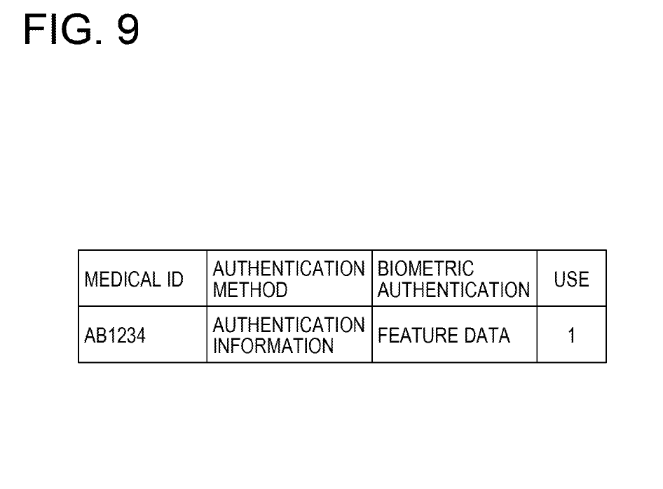

FIG. 9 illustrates an example of a table storing identity authentication information of a patient represented by the medical ID “AB0123” of FIG. 4. The table of FIG. 9 includes items of a medical ID of a patient, an authentication method, and authentication information. A description of information indicated by a flag of “use” is the same as the description of FIG. 5.

When the proxy is not a patient, proxy information 352 about the proxy may be recorded by using the table having the same forms as those of FIGS. 5, 8, and 9.

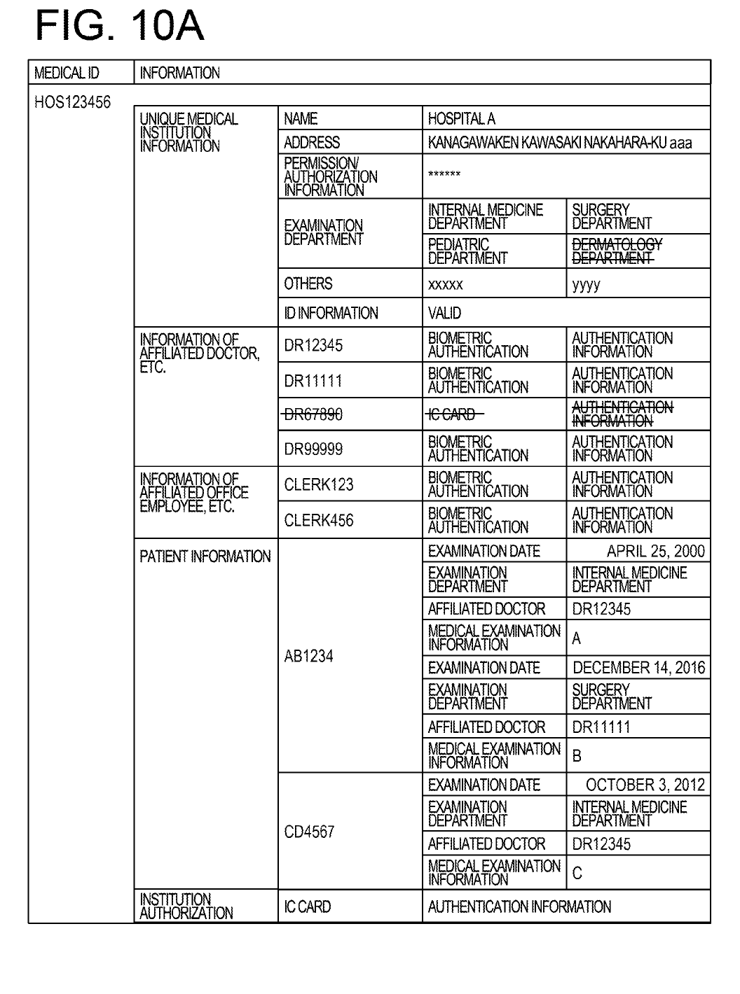

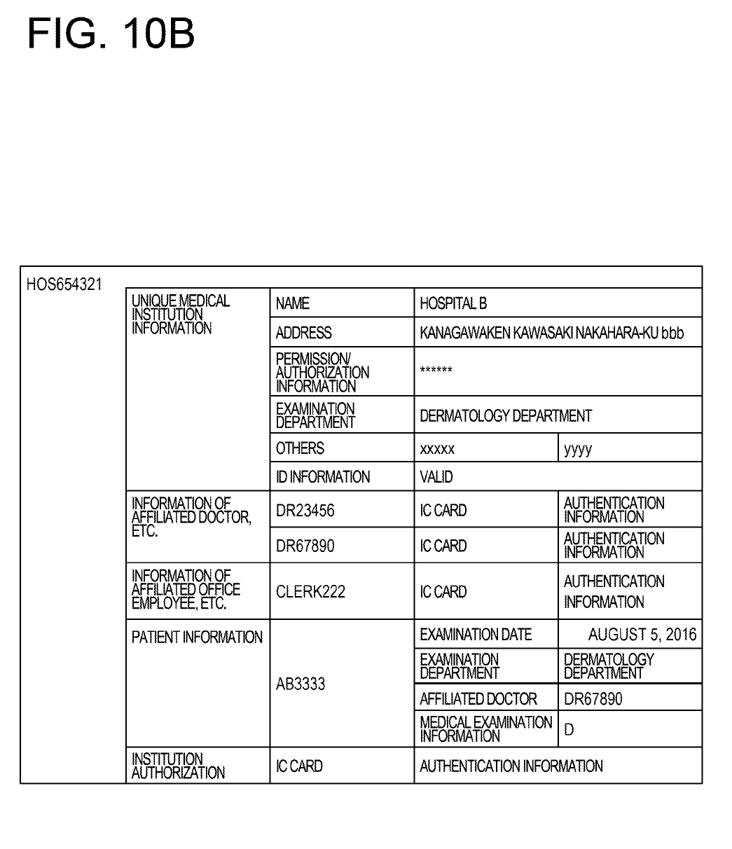

FIGS. 10A and 10B illustrate an example of the medical institution information 353-i. The information of FIGS. 10A and 10B includes information about hospital A indicated by the medical institution ID “HOS123456” and information about hospital B indicated by the medical institution ID “HOS654321”. The information about the hospital A corresponds to medical institution information 353-1, and the information about the hospital B corresponds to medical institution information 353-2. The information about each hospital includes items of unique medical institution information, information about an affiliated doctor and the like, information about an affiliated office employee, and the like, patient information, and institution authentication.

The unique medical institution information includes items of a name, an address, permission/authorization information, an examination department, others, and ID information of a medical institution. The ID information indicates whether a medical institution ID of a medical institution is effective. Information cancelled with a strike-out line among the examination departments of the hospital A indicates a past examination department, and information having no strike-out line indicates a present examination department.

The information about the affiliated doctors and the like includes items of a medical ID, an authentication method, and authentication information of a doctor belonging to the medical institution. Information cancelled with a strike-out line among the information about the affiliated doctor and the like of the hospital A indicates information about a doctor who was employed at the medical institution in the past, and information having no strike-out line indicates information about a doctor who presently works at the medical institution.

The information about the affiliated office employees and the like includes items of a medical ID, an authentication method, and authentication information of an office employee affiliated with the medical institution.

The information about the patient includes items of a medical examination date, an examination department, an affiliated doctor, medical examination information for each medical ID of a patient. The affiliated doctor is indicated by a medical ID of a doctor who is in charge of a patient, and the medical examination information indicates a result of a medical examination by an affiliated doctor. As described above, a medial ID of a patient and medical examination information of the patient are recorded in the medical institution information 353-i of FIGS. 10A and 10B in association with each other.

An authentication method and authentication information of a medical institution are recorded in the item of the institution authentication. For example, when the authentication method is an integrated circuit (IC) card, authentication data recorded in an IC card of the medical institution is recorded as authentication information.

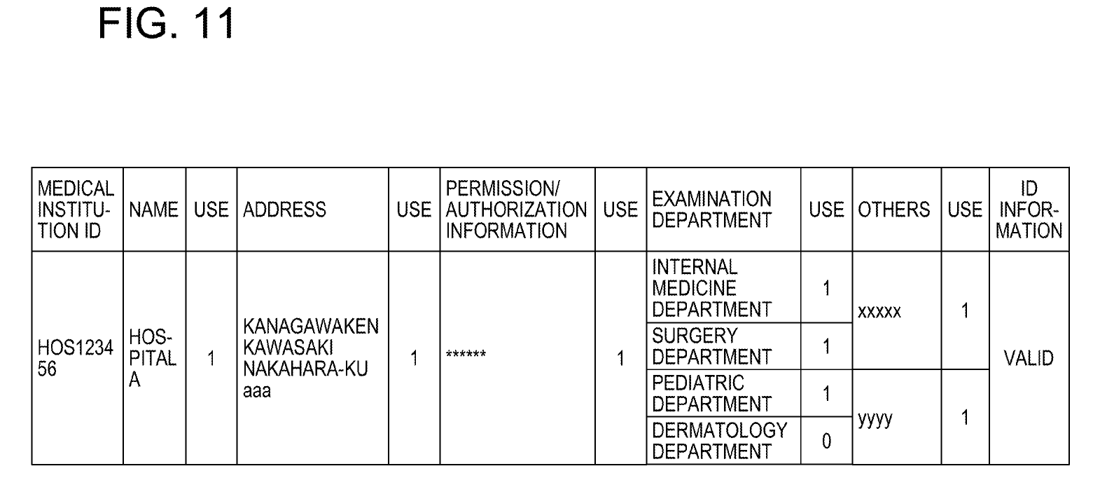

FIG. 11 illustrates an example of a table storing unique medical institution information of the hospital A indicated by the medical institution ID “HOS123456” of FIG. 10A. The table of FIG. 11 includes items of a medical institution ID, a name, an address, permission/authorization information, an examination department, others, and ID information of the hospital A. A description of information indicated by a flag of “use” is the same as the description of FIG. 5.

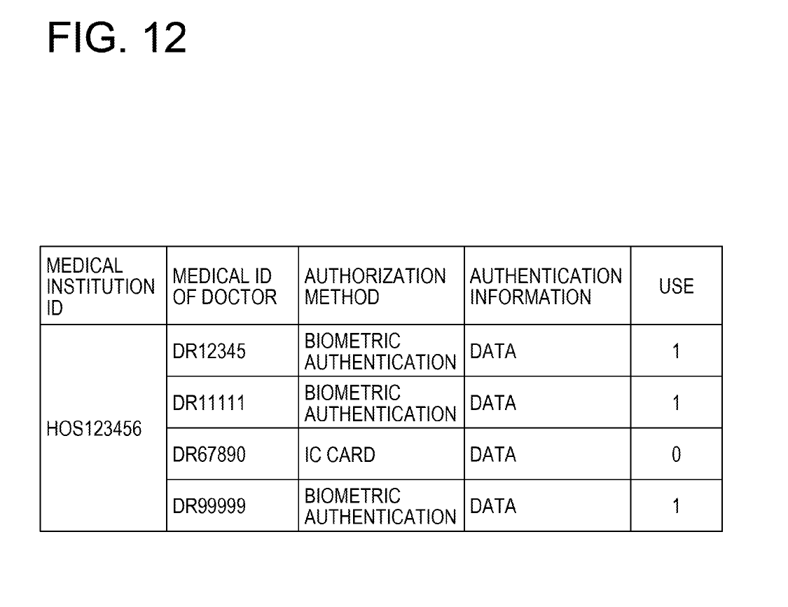

FIG. 12 illustrates an example of a table storing information, such as an affiliated doctor, of the hospital A of FIG. 10A. The table of FIG. 12 includes items of a medical institution ID, a medical ID of a doctor, an authentication method, and authentication information of the hospital A. A description of information indicated by a flag of “use” is the same as the description of FIG. 5.

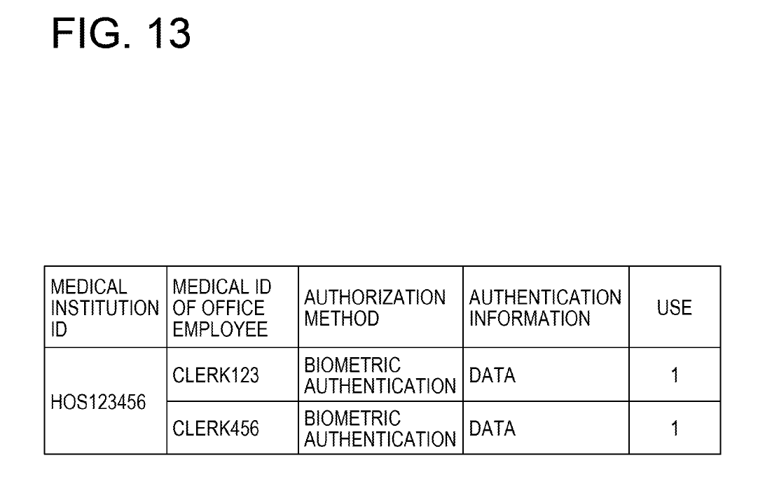

FIG. 13 illustrates an example of a table storing information, such as an affiliated office employee, of the hospital A of FIG. 10A. The table of FIG. 13 includes items of a medical institution ID, a medical ID of an office employee, an authentication method, and authentication information of the hospital A. A description of information indicated by a flag of “use” is the same as the description of FIG. 5.

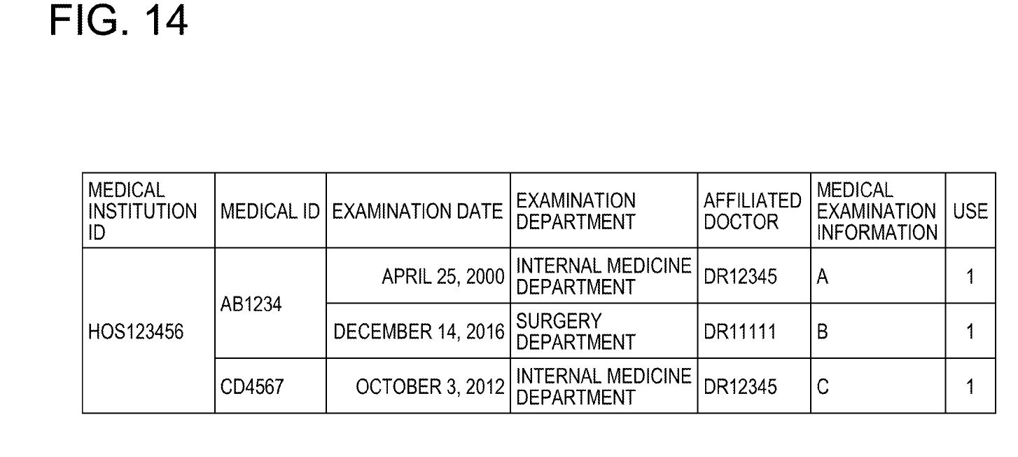

FIG. 14 illustrates an example of a table storing information about a patient of the hospital A of FIG. 10A. The table of FIG. 14 includes items of a medical institution ID, a medical ID of a patient, a medical examination date, an examination department, an affiliated doctor, and medical examination information of the hospital A. A description of information indicated by a flag of “use” is the same as the description of FIG. 5.

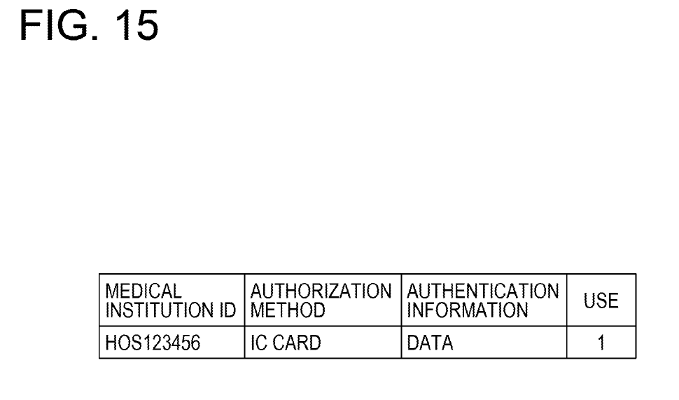

FIG. 15 illustrates an example of a table storing information of institution authentication of the hospital A of FIG. 10A. The table of FIG. 15 includes items of a medical institution ID, an authentication method, and authentication information of the hospital A. A description of information indicated by a flag of “use” is the same as the description of FIG. 5.

Whether to permit the disclosure of information is set in the disclosure permission information 343 stored in the server 322 for each item included in the medical institution information 353-i. For example, whether to permit the disclosure of information may be set for each of the plurality of elements of medical examination information of the same patient.

In the information processing system of FIG. 3, when the identity confirmation of a patient is available when the patient is examined, for example, information about the patient is obtained in a sequence described below.

(P1) In order to use the information processing system, a patient accesses the server 322 of the data center 304 by using the terminal device 301, and obtains a medical ID from the ID managing unit 341.

(P2) The patient writes the obtained medical ID in the recording medium 305, and the ID managing unit 341 writes the same medical ID in the patient information 351 of the storage device 324.

(P3) The patient presents the recording medium 305 during the examination in the hospital A, and an office employee inputs the medical ID recorded in the recording medium 305 to the terminal device 312-1 of the office employee.

(P4) The terminal device 312-1 performs identity authentication of the patient.

(P5) The terminal device 312-1 obtains information about the patient from the storage device 324 by using the medical ID of the patient. Accordingly, the patient information 351, the information about the patient included in the medical institution information 353-1 of the hospital A, and the information about the patient included in the medical institution information 353-2 of the hospital B are obtained.

(P6) The ID managing unit 341 connects the medical ID of the patient and the medical institution ID of the hospital A.

(P7) The ID managing unit 341 writes the medical institution ID of the hospital A in the examining medical institution included in the patient information 351.

(P8) A doctor examines the patient, and the ID managing unit 341 writes medical examination information and the like while associating the information about the patient included in the medical institution information 353-1 of the hospital A with the medical ID of the patient. In this case, the doctor receives a confirmation of whether to permit the disclosure of the medical examination information from the patient, and the ID managing unit 341 sets the disclosure or non-disclosure of the information in the disclosure permission information 343 according to an answer of the patient.

According to the foregoing information processing, on the premise of the permission of the disclosure of the information by the patient, the medical institution may refer to past medical examination information. The patient may determine whether to permit the disclosure of information for each item included in the medical institution information 353-i. Further, the medical ID of the patient is connected with the medical institution ID, so that the information about the patient may be obtained without combining the medical institution information 353-i of each medical institution.

In the meantime, in the information processing system of FIG. 3, when it is difficult to perform identity confirmation of a patient during a medical examination of the patient, for example, information about the patient is obtained in a sequence described below.

(P11) In order to use the information processing system, a patient accesses the server 322 of the data center 304 by using the terminal device 301, and obtains a medical ID from the ID managing unit 341.

(P12) The patient writes the obtained medical ID in the recording medium 305, and the ID managing unit 341 writes the same medical ID in the patient information 351 of the storage device 324.

(P13) The patient transmits a medical ID of a proxy recorded in the recording medium 306 of the proxy to the ID managing unit 341 and applies a registration of the proxy by using the terminal device 301.

(P14) The check unit 344 of the server 323 checks eligibility of the proxy.

(P15) When the proxy is eligible, the ID managing unit 341 writes the medical ID of the proxy in the patient information 351 of the storage device 324.

(P16) The ID managing unit 341 writes the medical ID of the patient in the proxy information 352.

(P17) When an unconscious patient is examined in the hospital A, the proxy presents the recording medium 306, and an office employee inputs the medical ID recorded in the recording medium 306 to the terminal device 312-1 of the office employee.

(P18) The terminal device 312-1 performs identity authentication of the proxy.

(P19) The terminal device 312-1 obtains information about a client from the proxy information 352 of the storage device 324 by using the medical ID of the proxy.

(P20) The office employee specifies the patient among the clients, and the terminal device 312-1 obtains information about the patient from the storage device 324 by using the medical ID of the specified patient. Accordingly, the patient information 351, the information about the patient included in the medical institution information 353-1 of the hospital A, and the information about the patient included in the medical institution information 353-2 of the hospital B are obtained.

(P21) The ID managing unit 341 connects the medical ID of the patient and the medical institution ID of the hospital A.

(P22) The ID managing unit 341 writes the medical institution ID of the hospital A in the examining medical institution included in the patient information 351.

(P23) A doctor examines the patient, and the ID managing unit 341 writes medical examination information and the like while associating the information about the patient included in the medical institution information 353-1 of the hospital A with the medical ID of the patient. In this case, the doctor receives a confirmation of whether to permit the disclosure of the medical examination information from the proxy, and the ID managing unit 341 sets the disclosure or non-disclosure of the information in the disclosure permission information 343 according to an answer of the proxy.

According to the foregoing information processing, even when it is difficult to perform the identity confirmation of the patient, the medical institution may refer to past medical examination information. In this case, the check unit 344 checks eligibility of the proxy, so that even though the medical institution does not perform a complicated operation, the medical institution may obtain medical examination information about the patient by an appropriate method.

The proxy may confirm a result of the medical examination and determine whether to permit a re-use of the medical examination information on behalf of the patient. For example, when an unconscious or older patient is examined, a family member and the like may respond to a medical examination inquiry, or receive an explanation of a result of the medical examination, and permit a re-use of the medical examination information instead of the patient.

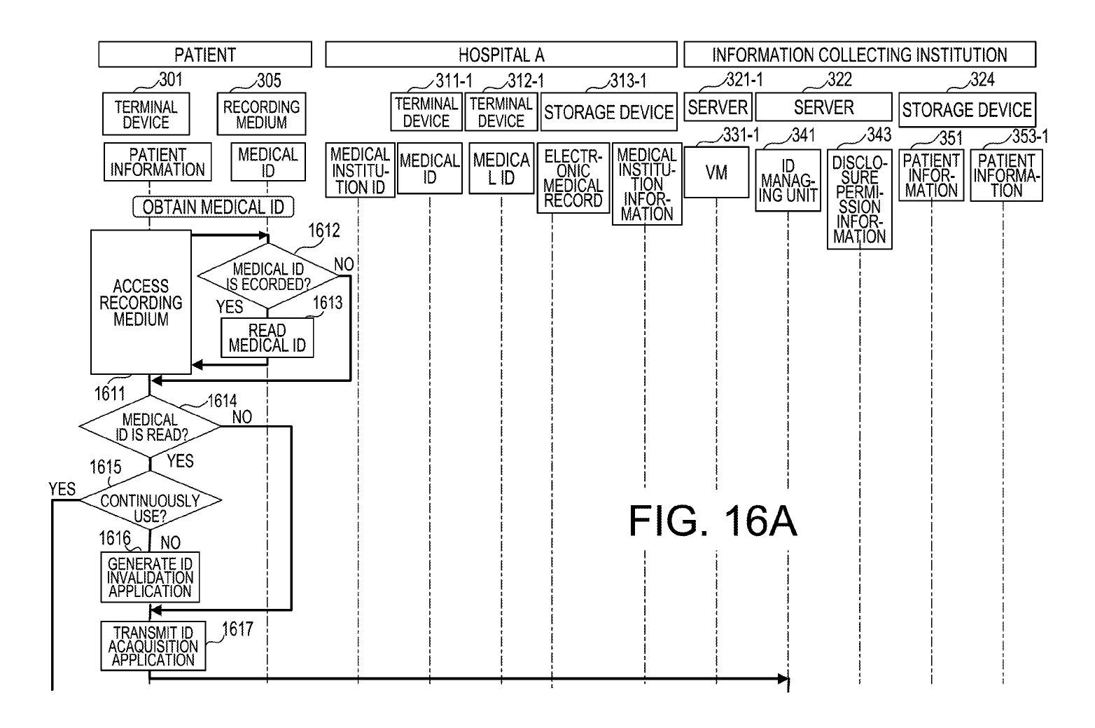

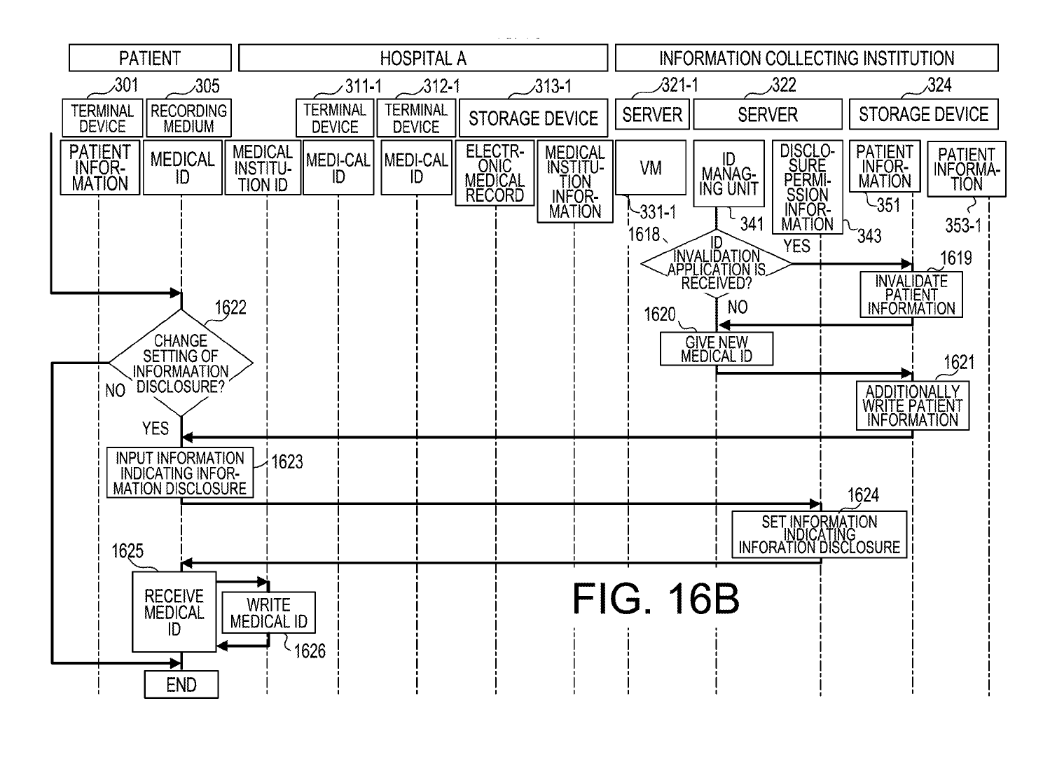

Next, an operation of the information processing system of FIG. 3 will be described in more detail with reference to FIGS. 16A and 16B to FIGS. 21A to 21H

FIGS. 16A and 16B illustrate an example of an ID obtaining sequence of obtaining, by a patient, a medical ID. First, a patient inputs patient information, such as a name, and a personal ID, such as my number, to the terminal device 301 and the terminal device 301 accesses the recording medium 305 by using the input information (step 1611). Then, the terminal device 301 checks whether an existing medical ID is recorded in the recording medium 305 (step 1612).

When the existing medical ID is recorded (step 1612, “YES”), the terminal device 301 reads the medical ID (step 1613). In the meantime, when the existing medial ID is not recorded (step 1612, “NO”), the terminal device 301 performs processing subsequent to step 1614.

Next, the terminal device 301 checks whether the existing medical ID is read from the recording medium 305 (step 1614). When the existing medical ID is read (step 1614, “YES”), the terminal device 301 inquires of the patient whether to continuously use the medical ID (step 1615).

When the patient inputs an answer indicating that the existing medical ID is not to be continuously used (step 1615, “NO”), the terminal device 301 generates an ID invalidation application that invalidates the existing medical ID (step 1616). Then, the terminal device 301 generates an ID acquisition application for obtaining a new medical ID, and transmits the ID invalidation application and the ID acquisition application to the ID managing unit 341 of the server 322 together with the patient information input by the patient (step 1617).

In the meantime, when the existing medical ID is not read (step 1614, “NO”), the terminal device 301 generates an ID acquisition application for obtaining a new medical ID, and transmits only the ID acquisition application to the ID managing unit 341 (step 1617).

The ID managing unit 341 checks whether the ID invalidation application is received (step 1618). When the ID invalidation application is received (step 1618, YES), the ID managing unit 341 invalidates all of the information associated with the existing medical ID included in the patient information 351 of the storage device 324 (step 1619). In this case, the ID managing unit 341 sets that the information is not used by setting “0” in the item of the use of the information.

In the meantime, when the ID invalidation application is not received (step 1618, “NO”), the terminal device 301 performs processing subsequent to step 1620.

Next, the ID managing unit 341 gives the new medical ID to the patient based on the received ID acquisition application (step 1620), and additionally writes the received patient information in the patient information 351 in association with the new medical ID (step 1621). Further, the ID managing unit 341 requests the setting of the information disclosure of the medical institution information 351-1 from the terminal device 301.

The patient inputs whether to permit the information disclosure for each item included in the medical institution information 353-1 by using the terminal device 301, and the terminal device 301 transmits the input information to the ID managing unit 341 (step 1623). The ID managing unit 341 sets the received information as disclosure permission information 343, and transmits the new medical ID to the terminal device 301 (step 1624). Then, the terminal device 301 receives the new medial ID (step 1625), and writes the received medical ID in the recording medium 305 (step 1626).

When the patient inputs an answer indicating that the existing medical ID is to be continuously used (step 1615, “YES”), the terminal device 301 inquires whether to change the setting of the information disclosure of the medical institution information 353-1 to the patient (step 1622). When the patient inputs an answer indicating that the setting of the information disclosure is to be changed (step 1622, “YES”), the terminal device 301 performs processing subsequent to step 1623. In this, processing of steps 1625 and 1626 is skipped.

In the meantime, when the patient inputs an answer indicating that the setting of the information disclosure is not to be changed (step 1622, “NO”), the terminal device 301 terminates the processing.

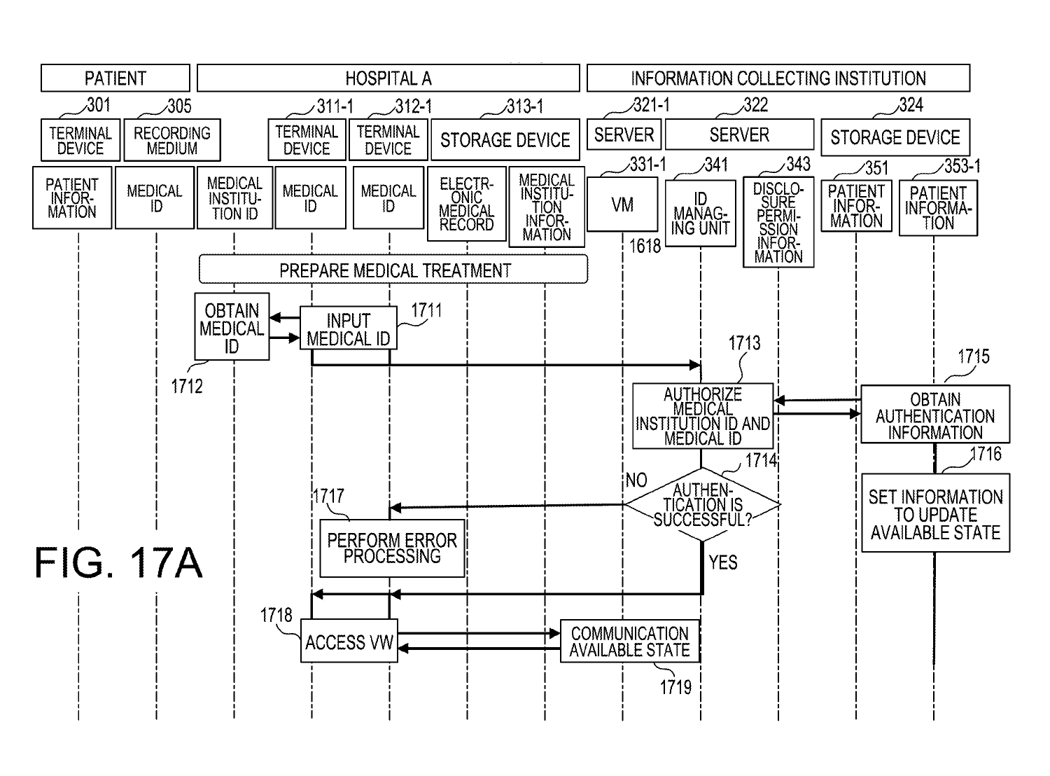

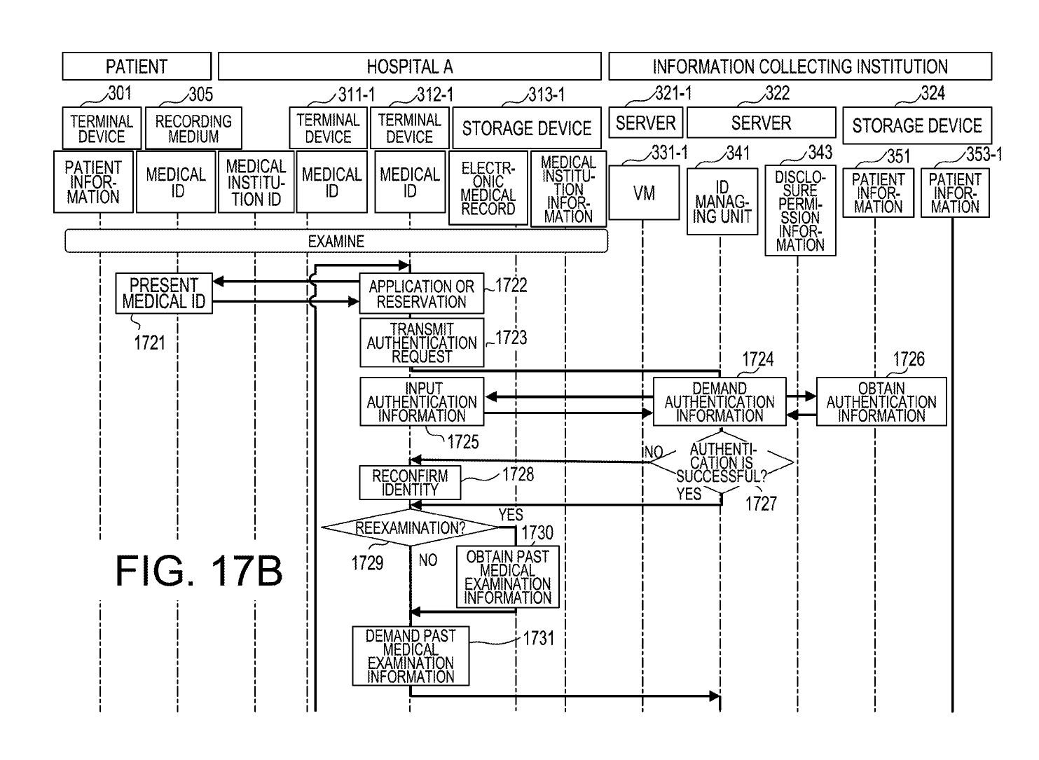

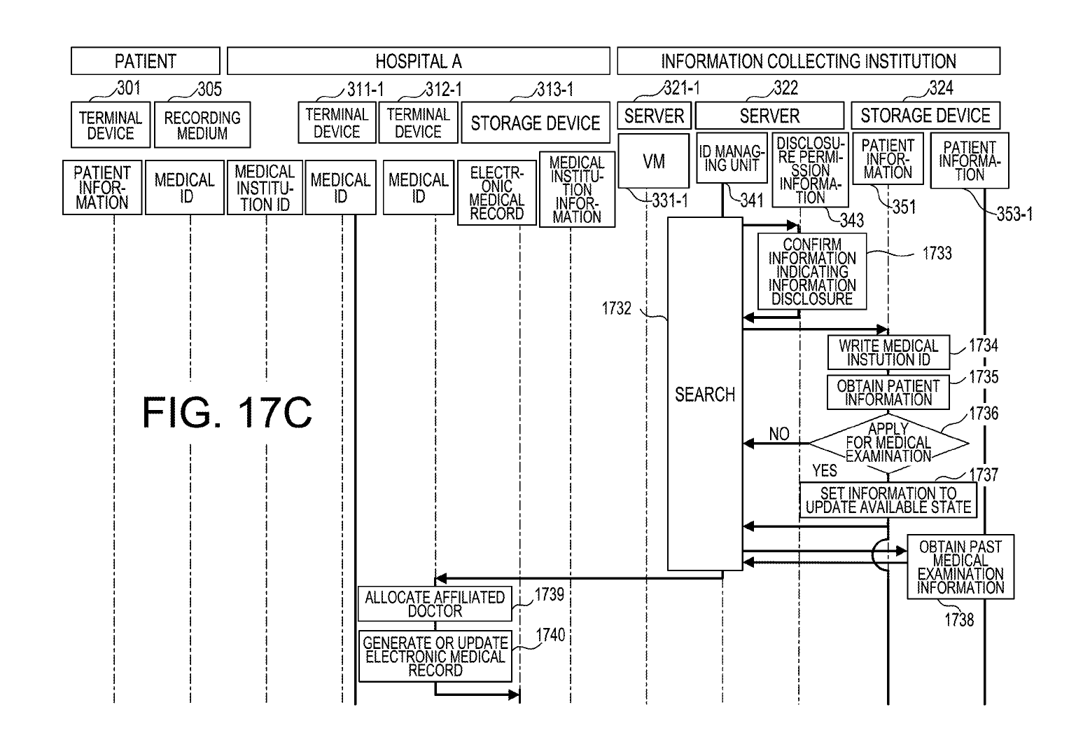

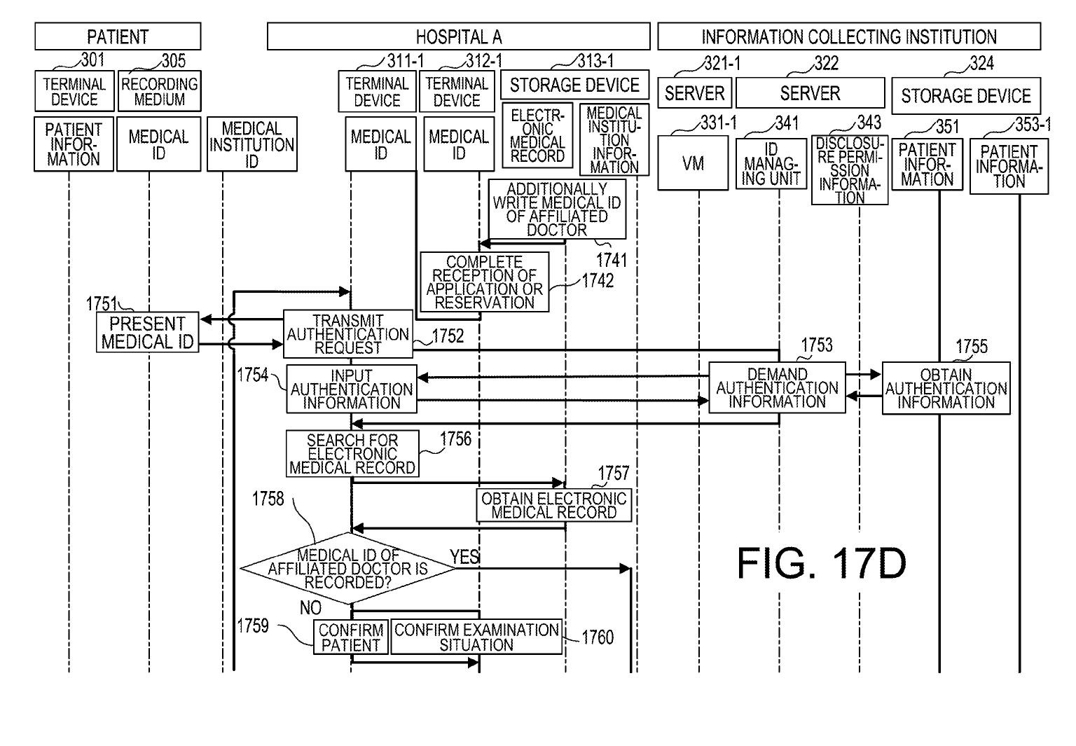

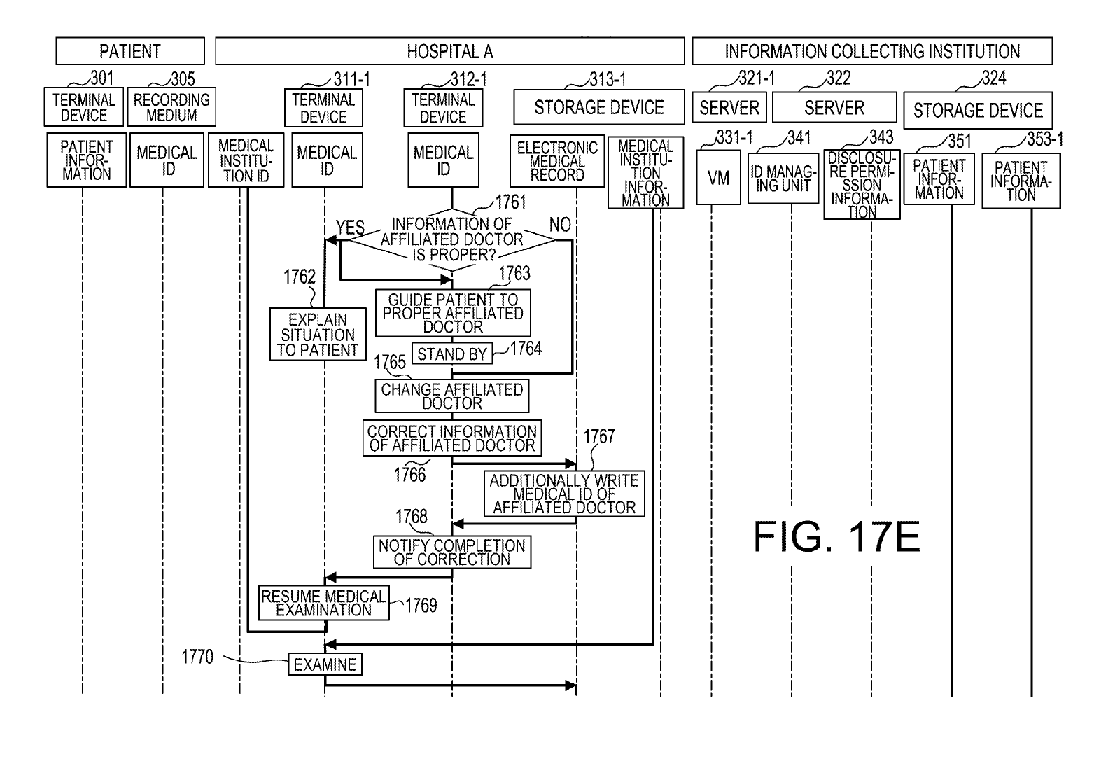

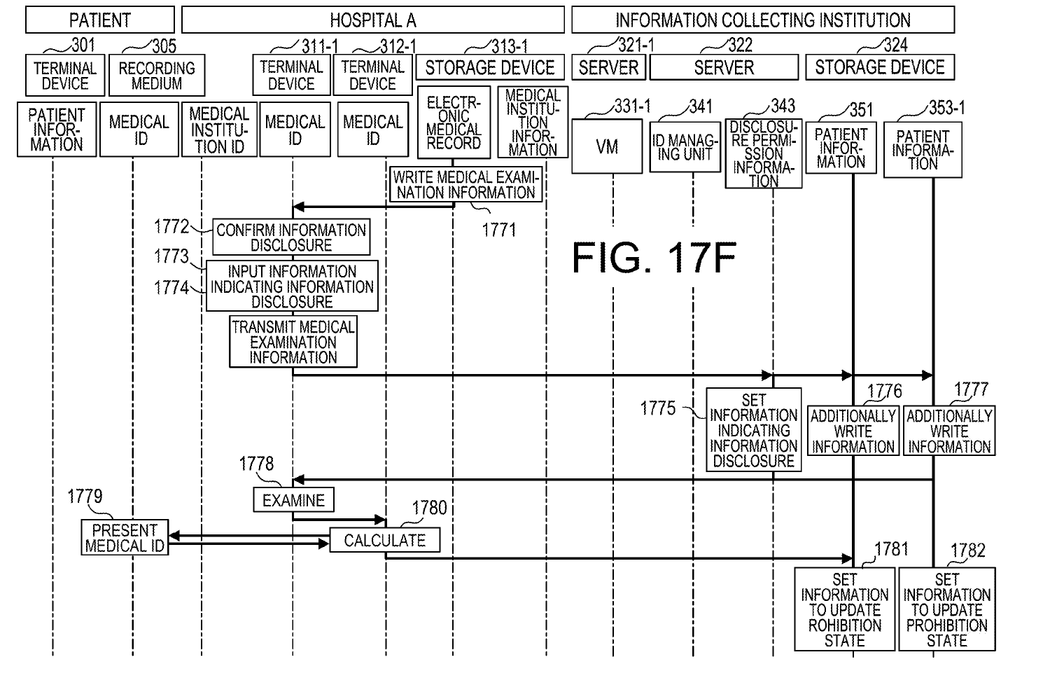

FIGS. 17A to 17F illustrate an example of a medical examination sequence when a patient is examined in the hospital A. In the present example, the terminal devices 311-1 and 312-1 of the hospital A store the medical institution ID and the authentication information of the hospital A. Further, the storage device 313-1 of the hospital A stores an electronic medical record including the medical examination information of the patient in the hospital A and the medical institution information of the hospital A.

First, before accepting a patient, a doctor of the hospital A inputs a medical ID and authentication information of the doctor to the terminal device 311-1 (step 1711). Then, the terminal device 311-1 obtains stored medical institution ID and authentication information of the hospital A (step 1712), and transmits an authentication request including the medical ID and the authentication information of the doctor, the medical institution ID and the authentication information of the hospital A to the ID managing unit 341.

Similarly, an office employee of the hospital A inputs a medical ID and authentication information of the office employee to the terminal device 312-1 (step 1711). Then, the terminal device 312-1 obtains stored medical institution ID and authentication information of the hospital A (step 1712), and transmits an authentication request including the medical ID and the authentication information of the office employee, the medical institution ID and the authentication information of the hospital A to the ID managing unit 341.

The ID managing unit 341 performs the authentication of the medical ID of the doctor and the medical institution ID of the hospital A included in the authentication request received from the terminal device 311-1 (step 1713), and checks whether the authentication is successful (step 1714).

In this case, the ID managing unit 341 obtains authentication information corresponding to the medical ID of the doctor and authentication information corresponding to the medical institution ID of the hospital A from the medical institution information 353-1 of the storage device 324 (step 1715). Then, for each of the medical ID and the medical institution ID, when the authentication information received from the terminal device 311-1 matches the authentication information obtained from the medical institution information 353-1, the ID managing unit 341 determines that the authentication is successful.

When the authentication is successful (step 1714, “YES”), the ID managing unit 341 sets the information about the hospital A included in the medical institution information 353-1 to an update available state (step 1716). For example, when an update flag of each medical institution is provided in the medical institution information 353-1, the ID managing unit 341 sets the information about the hospital A to an update available state by setting the update flag of the hospital A to be valid. Then, the ID managing unit 341 notifies the terminal device 311-1 of the authentication success.

The terminal device 311-1 accesses the VM 331-1 of the server 321-1 (step 1718), and the terminal device 311-1 and the VM 331-1 are in a communication available state (step 1719). Then, the terminal device 311-1 communicates with the ID managing 341 via the VM 331-1.

In the meantime, when the authentication fails (step 1714, “NO”), the ID managing unit 341 notifies the terminal device 311-1 of the authentication failure, and the terminal device 311-1 performs error processing (step 1717). In the error processing, the terminal device 311-1 performs processing subsequent to step 1711 again or terminates the processing.

Similarly, the ID managing unit 341 performs the authentication of the medical ID of the office employee and the medical institution ID of the hospital A included in the authentication request received from the terminal device 312-1 (step 1713), and checks whether the authentication is successful (step 1714).

In this case, the ID managing unit 341 obtains authentication information corresponding to the medical ID of the office employee and authentication information corresponding to the medical institution ID of the hospital A from the medical institution information 353-1 of the storage device 324 (step 1715). Then, for each of the medical ID and the medical institution ID, when the authentication information received from the terminal device 312-1 matches the authentication information obtained from the medical institution information 353-1, the ID managing unit 341 determines that the authentication is successful.

When the authentication is successful (step 1714, “YES”), the ID managing unit 341 sets the information about the hospital A included in the medical institution information 353-1 to an update available state (step 1716), and notifies the terminal device 312-1 of the authentication success. The terminal device 312-1 accesses the VM 331-1 of the server 321-1 (step 1718), and the terminal device 312-1 and the VM 331-1 are in a communication available state (step 1719). Then, the terminal device 312-1 communicates with the ID managing unit 341 via the VM 331-1.

In the meantime, when the authentication fails (step 1714, “NO”), the ID managing unit 341 notifies the terminal device 312-1 of the authentication failure, and the terminal device 312-1 performs error processing (step 1717). In the error processing, the terminal device 312-1 performs subsequent processing to step 1711 again or terminates the processing.

The patient presents the recording medium 305 at the reception when the patient is examined in the hospital A or notifies the medical ID of the patient through a phone and the like when making a reservation of a medical examination (step 1721). The office employee inputs the medical ID recorded in the recording medium 305, or the medical ID notified from the patient to the terminal device 312-1 (step 1722). Then, the terminal device 312-1 transmits an authentication request including the medical ID of the patient to the ID managing unit 341 (step 1723).

Next, the ID managing unit 341 demands the authentication information used for the identity authentication of the patient from the terminal device 312-1 (step 1724). Then, the patient inputs the authentication information to the terminal device 312-1 (step 1725), and the terminal device 312-1 transmits the input authentication information to the ID managing unit 341.

Next, the ID managing unit 341 obtains the authentication information corresponding to the medical ID of the patient from the patient information 351 of the storage device 324 (step 1726), and checks whether the authentication is successful (step 1727). For example, when the authentication information received from the terminal device 312-1 matches the authentication information obtained from the patient information 351, the ID managing unit 341 determines that the authentication is successful.

When the authentication fails (step 1727, “NO”), the ID managing unit 341 notifies the terminal device 312-1 of the authentication failure, and the office employee performs re-confirmation of an identity of the patient (step 1728).

In the meantime, when the authentication is successful (step 1727, “YES”), the ID managing unit 341 notifies the terminal device 312-1 of the authentication success. Then, the terminal device 312-1 checks whether the current examination is a re-examination (step 1729).

When the current examination is the re-examination (step 1729, “YES”), the terminal device 312-1 obtains past medical examination information of the patient from the electronic medical record of the storage device 313-1 (step 1730). Then, the terminal device 312-1 transmits an information obtaining request including the medical ID of the patient and the medical institution ID of the hospital A to the ID managing unit 341, and demands past medical examination information at another hospital (step 1731).

In the meantime, when the current examination is a first examination (step 1729, “NO”), the terminal device 312-1 performs processing subsequent to step 1731.

Next, the ID managing unit 341 searches for medical examination information corresponding to the medical ID of the patient in the storage device 324 (step 1732). First, the ID managing unit 341 obtains information corresponding to the medical ID of the patient from the disclosure permission information 343, and checks the item of which the information disclosure is permitted (step 1733).

Next, the ID managing unit 341 writes the medical institution ID of the hospital A in the item of the examining medical institution corresponding to the medical ID of the patient of the patient information 351 (step 1734). Then, the ID managing unit 341 obtains basic patient information and basic medical information corresponding to the medical ID of the patient from the patient information (step 1735).

Next, the ID managing unit 341 checks whether a chance of the authentication request is an application for the medical examination (step 1736). When the chance of the authentication request is the application for the medical examination (step 1736, “YES”), the ID managing unit 341 sets the information about the patient included in the patient information 351 to an update available state (step 1737). For example, when an update flag of each patient is provided in the patient information 351, the ID managing unit 341 sets the information of the patient to an update available state by setting an update flag of the patient receiving the medical examination to be valid.

In the meantime, when the chance of the authentication request is a reservation of the medical examination (step 1736, “NO”), the ID managing unit 341 performs processing subsequent to step 1738.

Next, the ID managing unit 341 obtains past medical examination information corresponding to the medical ID of the patient from the medical institution information 353-i (step 1738). Accordingly, the medical examination information of which the information disclosure is permitted among the plurality of elements of the medical examination information included in the medical institution information 353-1 of the hospital A and the medical institution information 353-2 of the hospital B is obtained. Further, the ID managing unit 341 transmits the information obtained from the patient information 351 and the medical institution information 353-i to the terminal device 312-1.

Next, the office employee allocates an affiliated doctor to the patient (step 1739) and the terminal device 312-1 generates or updates the electronic medical record of the patient (step 1740). Accordingly, the information received from the ID managing unit 341 and the medical ID of the allocated affiliated doctor are additionally written in the electronic medical record (step 1741). Further, the terminal device 312-1 completes the reception of the application or the reservation of the patient (step 1742), and processing subsequent to step 1722 is repeated for a next patient.

In an examination room, the patient presents the recording medium 305 to the doctor (step 1751), and the doctor inputs the medical ID recorded in the recording medium 305 and the medical ID of the doctor to the terminal device 311-1 (step 1752). Then, the terminal device 311-1 transmits an authentication request including the medical ID of the patient to the ID managing unit 341.

Next, the ID managing unit 341 demands the authentication information used for the identity authentication of the patient from the terminal device 311-1 (step 1753). Then, the patient inputs the authentication information to the terminal device 311-1 (step 1754), and the terminal device 311-1 transmits the input authentication information to the ID managing unit 341

Next, the ID managing unit 341 obtains the authentication information corresponding to the medical ID of the patient from the patient information 351 of the storage device 324 (step 1755), and performs identity authentication of the patient by using the obtained authentication information and the authentication information received from the terminal device 311-1.

Next, the terminal device 311-1 searches for the electronic medical record corresponding to the medical ID of the patient in the storage device 313-1 (step 1756), and obtains the electronic medical record (step 1757). Then, the terminal device 311-1 checks whether the medical ID of the doctor is recorded in the obtained electronic medical record (step 1758).

When the medical ID of the doctor is not recorded in the electronic medical record (step 1758, “NO”), the terminal device 311-1 requests the confirmation of an examination situation from the terminal device 312-1. The doctor confirms the patient (step 1759), and the office employee confirms the examination situation (step 1760). Then, the office employee checks whether the information of the affiliated doctor recorded in the electronic medical record is proper (step 1761).

When the information of the affiliated doctor is proper (step 1761, “YES”), there is a possibility that the patient may make a mistake of an incorrect examination room. Accordingly, the doctor explains the situation to the patient (step 1762), and the office employee guides the patient to an examination room of the proper affiliated doctor (step 1763), and the patient stands by in the guided examination room (step 1764).

In the meantime, when the information of the affiliated doctor is not proper (step 1761, “NO”), the office employee changes an affiliated doctor (step 1765), and the terminal device 312-1 corrects the information about the affiliated doctor recorded in the electronic medical record (step 1766). Accordingly, a medical ID of the corrected affiliated doctor is additionally written in the electronic medical record (step 1767).

Next, the terminal device 312-1 notifies the terminal device 311-1 of the completion of the correction (step 1768), and the doctor resumes an examination of the patient (step 1769), and the terminal device 311-1 repeats processing subsequent to step 1752.

When the medical ID of the doctor is recorded in the electronic medical record (step 1758, “YES”), the doctor examines the patient, and inputs medical examination information indicating a result of the examination to the terminal device 311-1 (step 1770). The medical examination information may include a prescription and the like. Then, the terminal device 311-1 writes the medical examination information in the electronic medical record of the storage device 313-1 (step 1771).

Next, the doctor confirms whether to permit the disclosure of the medical examination information to the patient (step 1772), and inputs the disclosure or non-disclosure of the information to the terminal device 311-1 based on an answer of the patient (step 1773). Then, the terminal device 311-1 transmits the medical ID of the patient, the medical ID of the doctor, the medical examination information, and the information indicating the disclosure or non-disclosure of the information to the ID managing unit 341 (step 1774).

The ID managing unit 341 sets the disclosure or non-disclosure of the information in the disclosure permission information 343 based on the information received from the terminal device 311-1 (step 1775). Next, the ID managing unit 341 additionally writes an examination date in the item of the examining medical institution corresponding to the medical ID of the patient of the patient information 351 (step 1776). Further, the ID managing unit 341 additionally writes the examination date, the examination department, the affiliated doctor, and the medical examination information in the information about the patient corresponding to the medical ID of the patient of the medical institution information 353-1 (step 1777).

The doctor terminates the examination (step 1778), the patient presents the recording medium 305 at the reception (step 1779), and the office employee inputs the medical ID recorded in the recording medium 305 to the terminal device 312-1, and performs a calculation (step 1780). Then, the terminal device 312-1 notifies the ID managing unit 341 of the termination of the medical examination.

Next, the ID managing unit 341 sets the information about the patient included in the patient information 351 to an update prohibition state (step 1781). For example, the ID managing unit 341 sets an update flag of the examined patient to be invalid, so that the information about the patient is set to an update prohibition state. Further, the ID managing unit 341 sets the information about the hospital A included in the medical institution information 353-1 to the update prohibition state (step 1782). For example, the ID managing unit 341 sets an update flag of the hospital A to be invalid, so that the information about the hospital A is set to the update prohibition state.

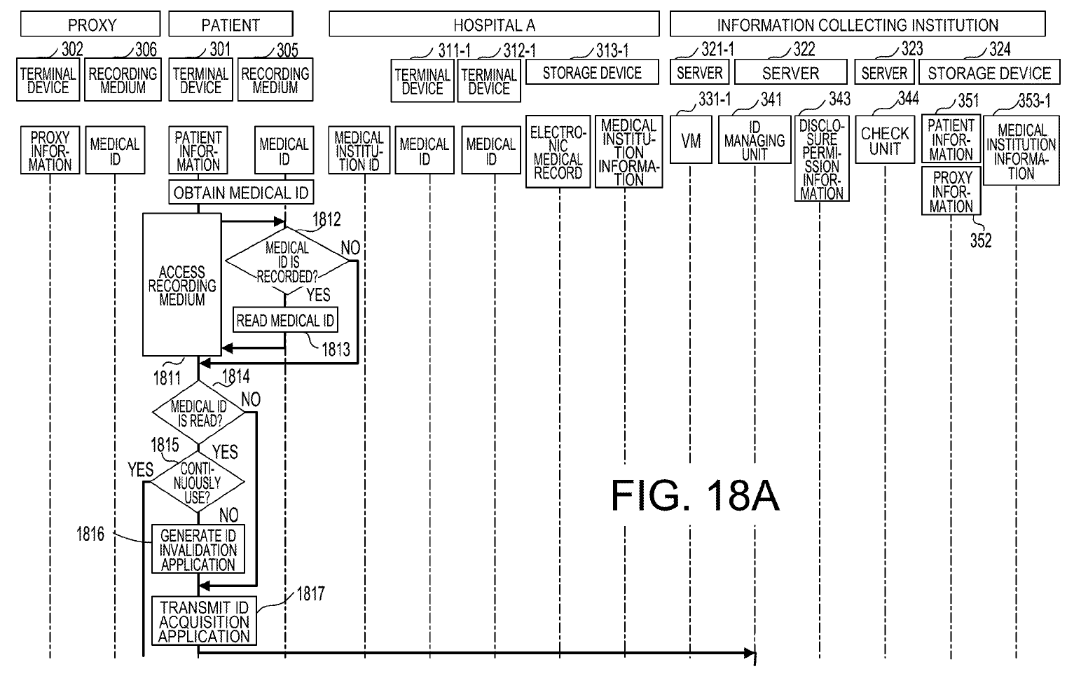

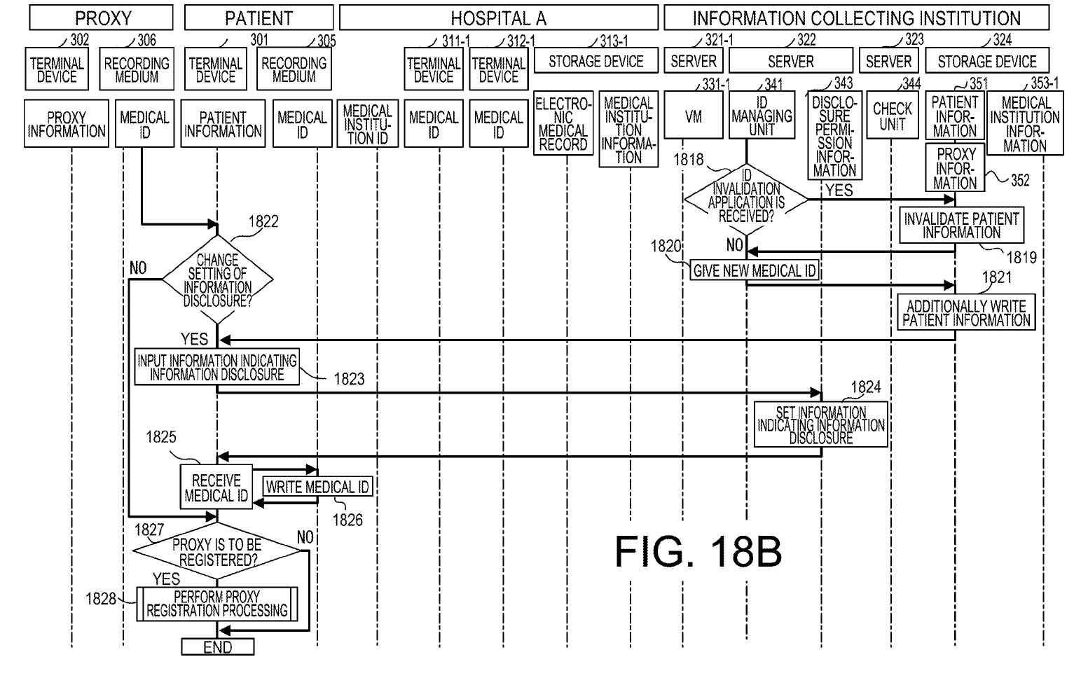

FIGS. 18A and 18B illustrate an example of an ID obtaining sequence of obtaining a medical ID of a patient and registering a proxy. Processing of steps 1811 to 1826 is the same as the processing of step 1611 of FIG. 16A to step 1626 of FIG. 16B.

Next, the terminal device 301 inquires of a patient whether to register a proxy (step 1827). When the patient inputs an answer indicating that the proxy is to be registered (step 1827, “YES”), the terminal device 301 performs proxy registration processing (step 1828). In the meantime, when the patient inputs an answer indicating that the proxy is not to be registered (step 1827, “NO”), the terminal device 301 terminates the processing.

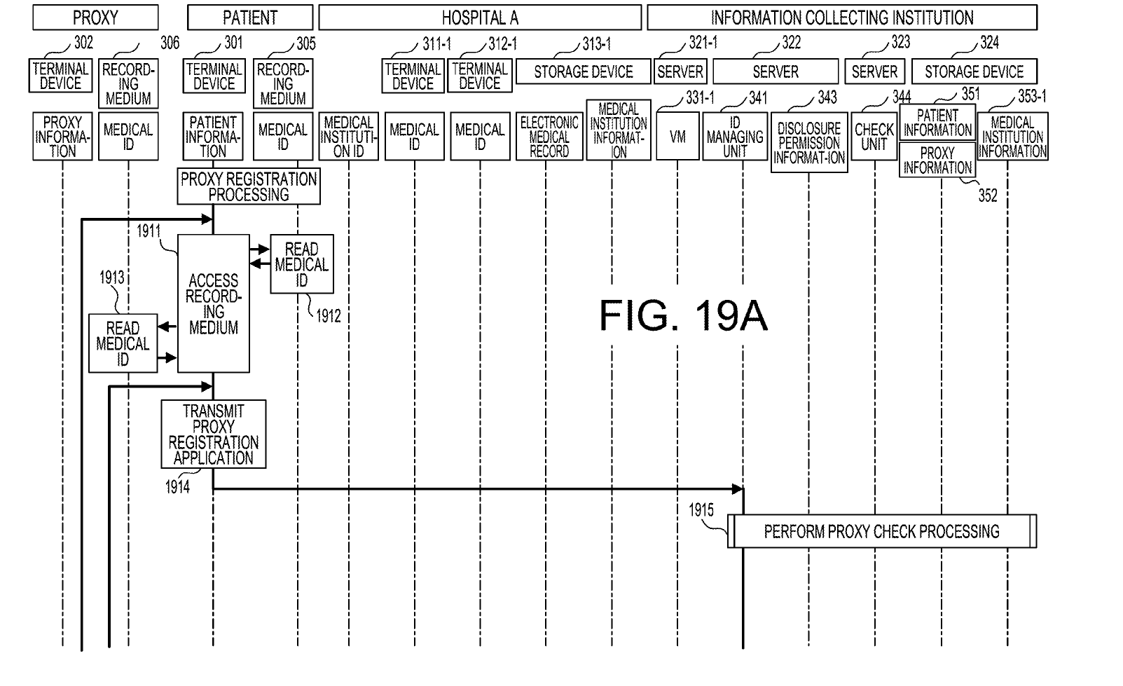

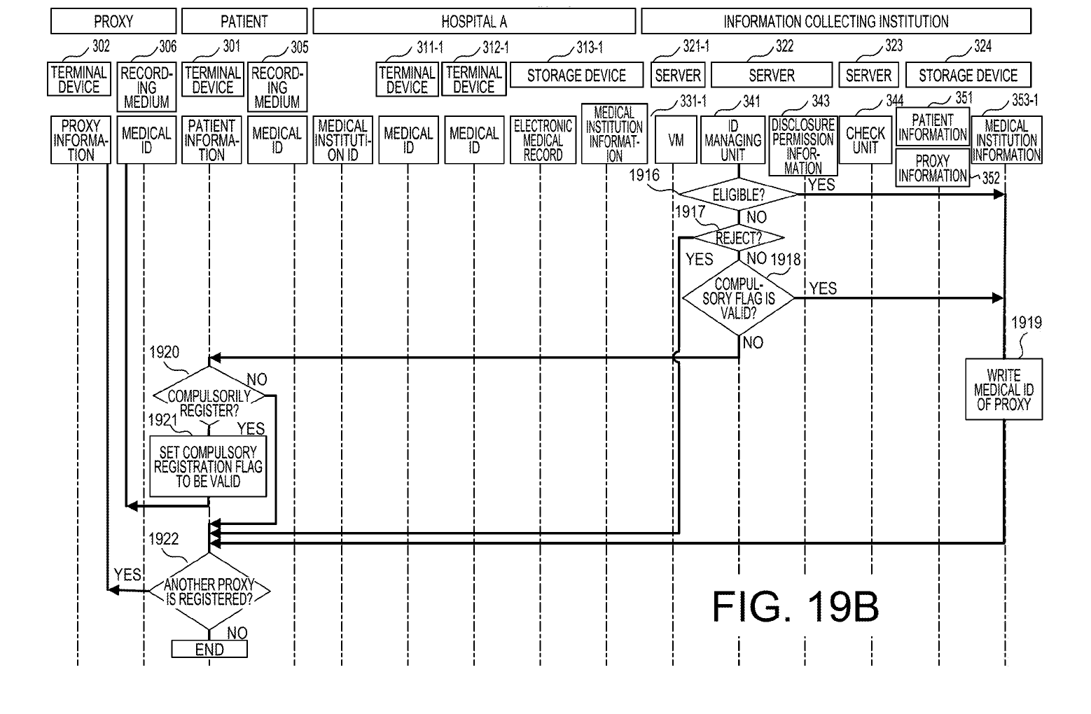

FIGS. 19A and 19B illustrate an example of the proxy registration processing in step 1828 of FIG. 18B. First, the patient inputs patient information, such as a name of a patient, and a personal ID, such as my number, to the terminal device 301 and the terminal device 301 accesses the recording medium 305 of the patient by using the input information (step 1911). Then, the terminal device 301 reads the medical ID of the patient from the recording medium 305 (step 1912).

Next, the patient inputs proxy information, such as a name of the proxy, and a personal ID, such as my number, to the terminal device 301 and the terminal device 301 accesses the recording medium 306 of the proxy by using the input information. Then, the terminal device 301 reads the medical ID of the proxy from the recording medium 305 (step 1913).

Next, the terminal device 301 transmits the medical ID of the patient, the medical ID of the proxy, and a proxy registration application including a compulsory registration flag to the ID managing unit 341 (step 1914).

The ID managing unit 341 performs proxy check processing based on the received proxy registration application (step 1915). Then, the ID managing unit 341 determines whether the applied proxy is eligible based on a result of the proxy check processing (step 1916).

When the proxy is eligible (step 1916, “YES”), the ID managing unit 341 registers the medical ID of the proxy in the information about the patient corresponding to the medical ID of the patient included in the proxy registration application in the patient information 351 (step 1919). Then, the ID managing unit 341 notifies the terminal device 301 of the completion of the registration.

Next, the terminal device 301 inquires of the patient whether to register another proxy (step 1922). When the patient inputs an answer indicating that another proxy is to be registered (step 1922, “YES”), the terminal device 301 repeats processing subsequent to step 1911 for another proxy. In the meantime, when the patient inputs an answer indicating that another proxy is not to be registered (step 1922, “NO”), the terminal device 301 terminates the processing.

When the proxy is not eligible (step 1916, “NO”), the ID managing unit 341 determines whether the applied proxy is rejected based on the result of the proxy check processing (step 1917). When the proxy is rejected (step 1917, “YES”), the ID managing unit 341 notifies the terminal device 301 of the rejection of the registration. Then, the terminal device 301 performs processing subsequent to step 1922.

In the meantime, when the proxy is not rejected (step 1917, “NO”), the ID managing unit 341 checks whether the compulsory registration flag included in the proxy registration application is valid (step 1918). When the compulsory registration flag is invalid (step 1918, “NO”), the ID managing unit 341 notifies the terminal device 301 of suspension of the registration.

Next, the terminal device 301 inquires of the patient whether to compulsorily register a proxy (step 1920). When the patient inputs an answer indicating that the proxy is to be compulsorily registered (step 1920, “YES”), the terminal device 301 sets the compulsory registration flag to be valid (step 1921) and repeats processing subsequent to step 1914. In this case, for the same proxy, processing of steps 1915 to 1918 is performed again.

When the compulsory registration flag is valid (step 1918, “YES”), the ID managing unit 341 registers a medical ID of the proxy in the information about the patient corresponding to the medical ID of the patient (step 1919). Then, the ID managing unit 341 notifies the terminal device 301 of the completion of the registration.

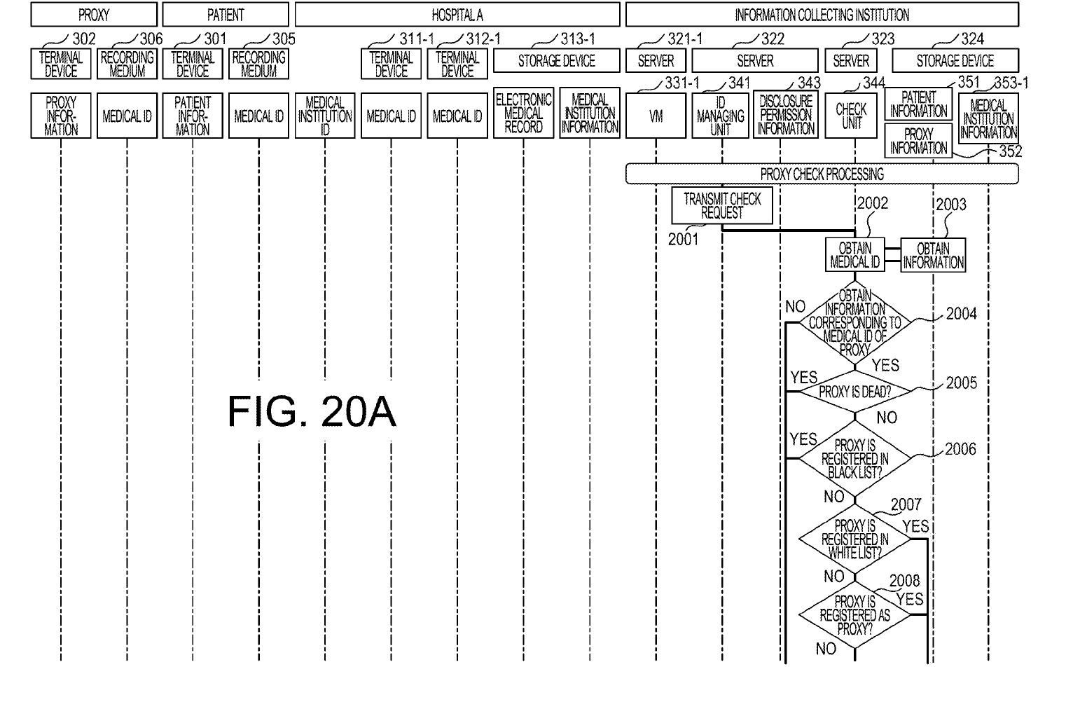

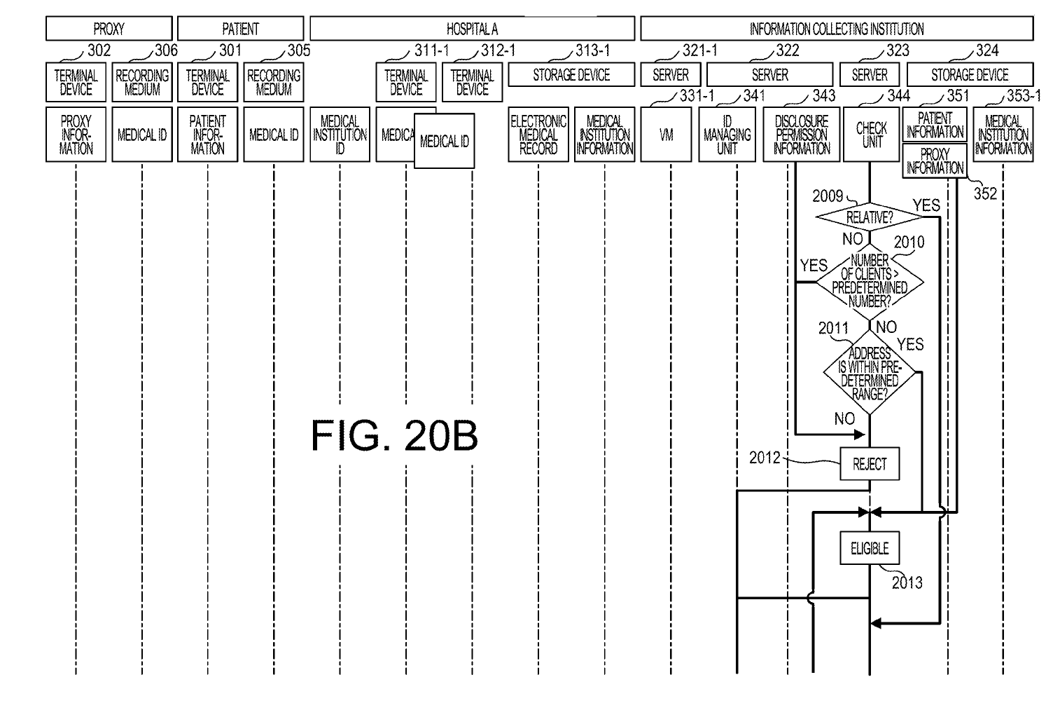

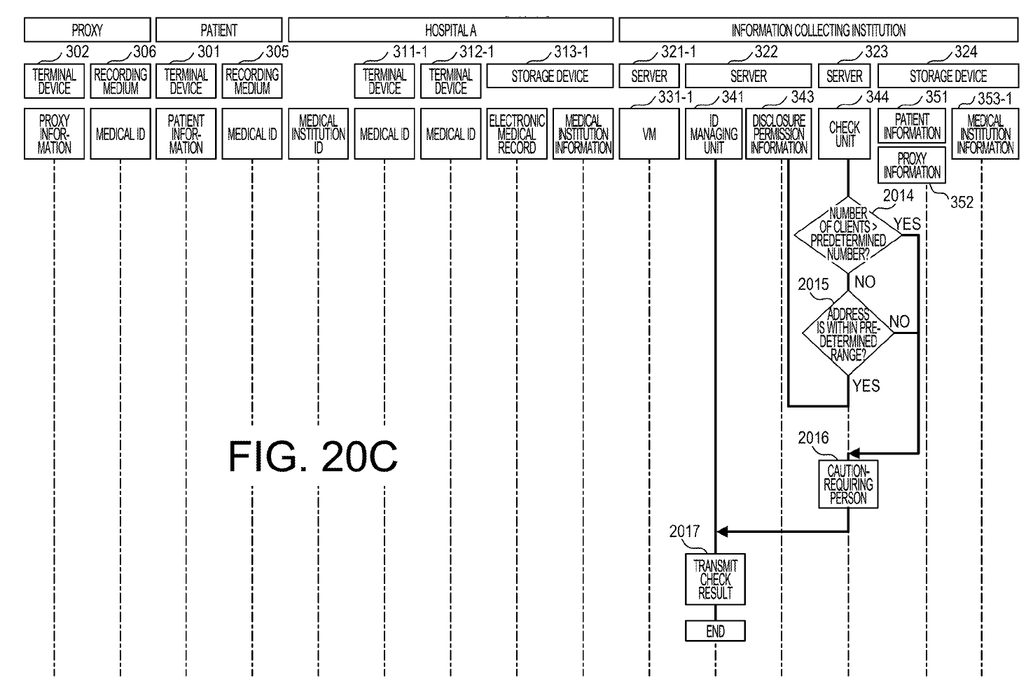

FIGS. 20A to 20C illustrate an example of the proxy check processing in step 1915 of FIG. 19A. First, the ID managing unit 341 obtains the medical ID of the patient and the medical ID of the proxy from the proxy registration application, and transmits a check request including the IDs to the check unit 344 of the server 323 (step 2001).

The check unit 344 obtains the medical ID of the patient and the medical ID of the proxy from the received check request (step 2002). Then, the check unit 344 obtains information corresponding to the medical ID of the patient from the patient information 351 of the storage device 324, and obtains information corresponding to the medical ID of the proxy from the proxy information 352 (step 2003).

Next, the check unit 344 checks whether the information corresponding to the medical ID of the proxy is obtained (step 2004). When the information corresponding to the medical ID of the proxy does not exist (step 2004, “NO”), the check unit 344 generates a check result indicating that the proxy is rejected (step 2012). Then, the check unit 344 transmits the generated check result to the ID managing unit 341 (step 2017).

In the meantime, when the information corresponding to the medical ID of the proxy is obtained (step 2004, “YES”), the check unit 344 checks whether the proxy is dead by referring to the item, the life or death of the basic medical information of the proxy (step 2005). When the proxy is dead (step 2004, “YES”), the check unit 344 performs processing subsequent to step 2012.

In the meantime, when the proxy is not dead (step 2004, “NO”), the check unit 344 checks whether the proxy is registered in a black list (step 2006). The black list is a list of an undesirable person as a proxy representing a patient, and for example, the black list is written by the information collecting institution and is stored in the server 323. When the proxy is registered in the black list (step 2006, “YES”), the check unit 344 performs processing subsequent to step 2012.

In the meantime, when the proxy is not registered in the black list (step 2006, “NO”), the check unit 344 checks whether the proxy is registered in a white list (step 2007). The white list is a list of a desirable person as a proxy representing a patient, and for example, the white list is written by the information collecting institution and is stored in the server 323.

When the proxy is registered in the white list (step 2007, “YES”), the check unit 344 generates a check result indicating that the proxy is eligible (step 2013). Then, the check unit 344 transmits the generated check result to the ID managing unit 341 (step 2017).

In the meantime, when the proxy is not registered in the white list (step 2007, “NO”), the check unit 344 checks whether the medical ID of the proxy has been already registered by referring to the item of the proxy in the information about the patient (step 2008). When the medical ID of the proxy has been already registered (step 2008, “YES”), the check unit 344 performs processing subsequent to step 2013.

In the meantime, when the medical ID of the proxy is not registered (step 2008, “NO”), the check unit 344 checks whether the patient and the proxy are in a predetermined relative relation (step 2009). The predetermined relative relation may be a relative regulated under the Civil Law, and may be a narrower range or a wider range than the relative regulated under the Civil Law. For example, the predetermined relative relation may be limited only to a blood relationship within the predetermined degree of kinship and a spouse.

The check unit 344 may obtain information indicating whether the patient and the proxy are in the predetermined relative relation by inquiring a family register relation between the patient and the proxy to a server (not illustrated) which manages family register information about the patient and the proxy.

When the patient and the proxy are not in the predetermined relative relation (step 2009, “NO”), the check unit 344 checks whether the number of clients who the proxy acts on behalf of is larger than a predetermined number (step 2010). In this case, the check unit 344 counts the number of currently used medical IDs of the clients by referring to the item of the client of the information about the proxy, and uses the number of counted medical IDs as the number of clients. When the number of clients is larger than the predetermined number (step 2010, “YES”), the check unit 344 performs processing subsequent to step 2012.

In the meantime, when the number of clients is equal to or smaller than the predetermined number (step 2010, “NO”), the check unit 344 checks whether an address of the proxy is within a predetermined range based on the address of the patient (step 2011). For example, the predetermined range may be a range of the same prefecture or the same regional category.

When the address of the proxy is within the predetermined range (step 2011, “YES”), the check unit 344 performs processing subsequent to step 2013. In the meantime, when the address of the proxy is not within the predetermined range (step 2011, “NO”), the check unit 344 performs processing subsequent to step 2012.

When the patient and the proxy are in the predetermined relative relation (step 2009, “YES”), the check unit 344 checks whether the number of clients who the proxy acts on behalf of is larger than the predetermined number identically to step 2010 (step 2014). When the number of clients is larger than the predetermined number (step 2014, “YES”), the check unit 344 generates a check result indicating that the proxy is a caution-requiring person (step 2016). Then, the check unit 344 transmits the generated check result to the ID managing unit 341 (step 2017).

In this case, the result of the determination of step 1916 of FIG. 19B is “NO” and the result of the determination of step 1917 is also “NO,” so that whether the compulsory registration flag is valid is checked in step 1918.

In the meantime, when the number of clients is equal to or smaller than the predetermined number (step 2014, “NO”), the check unit 344 checks whether the address of the proxy is within the predetermined range identically to step 2011 (step 2015). When the address of the proxy is not within the predetermined range (step 2015, “NO”), the check unit 344 performs processing subsequent to step 2016. In the meantime, when the address of the proxy is within the predetermined range (step 2015, “YES”), the check unit 344 performs processing subsequent to step 2013.

As described above, the eligibility of the proxy is checked based on the patient information 351 and the proxy information 352, thereby preventing an ineligible proxy from being registered. For example, when one proxy acts on behalf of a plurality of patients, it is considered that the proxy is not an ordinary person, but is ineligible as the proxy. Further, even though the number of clients is small, the addresses of the clients of the proxy are distributed all over the country, the proxy is also considered ineligible.

The check of the proxy based on the list of the list may be preferentially performed compared to the check based on the number of clients and the address of the client by using the black list or the white list.

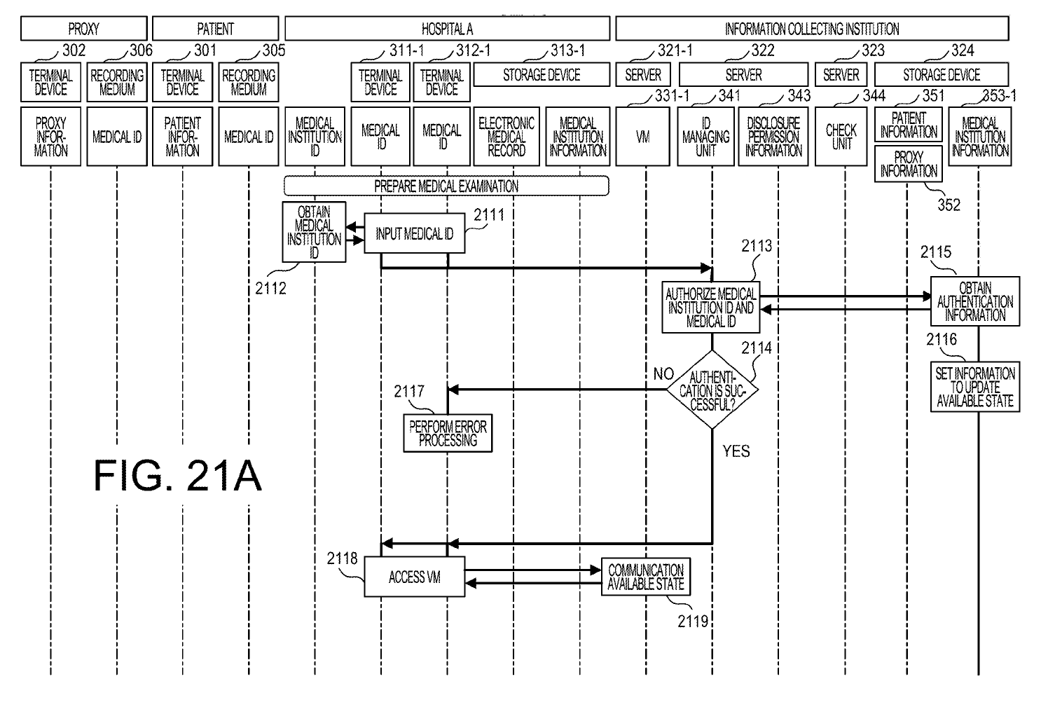

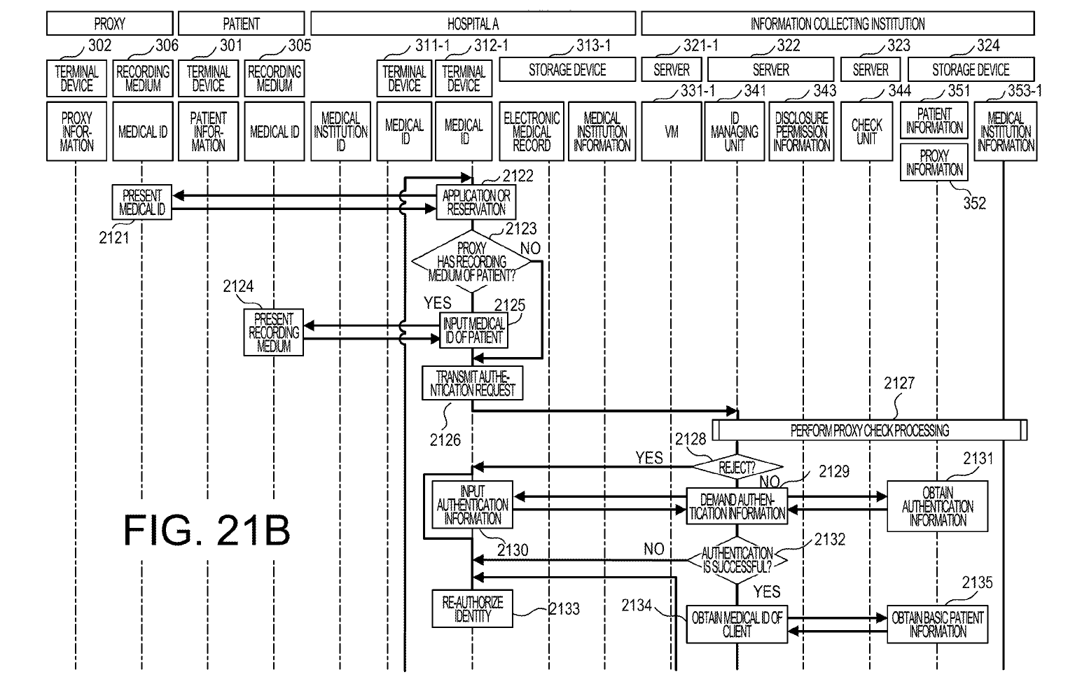

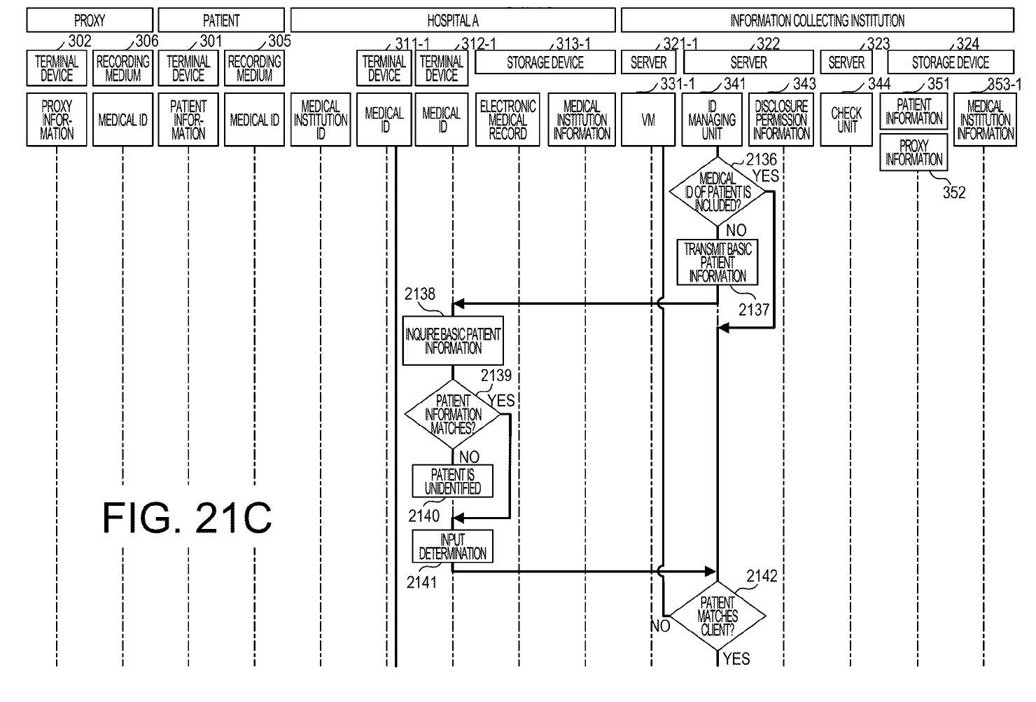

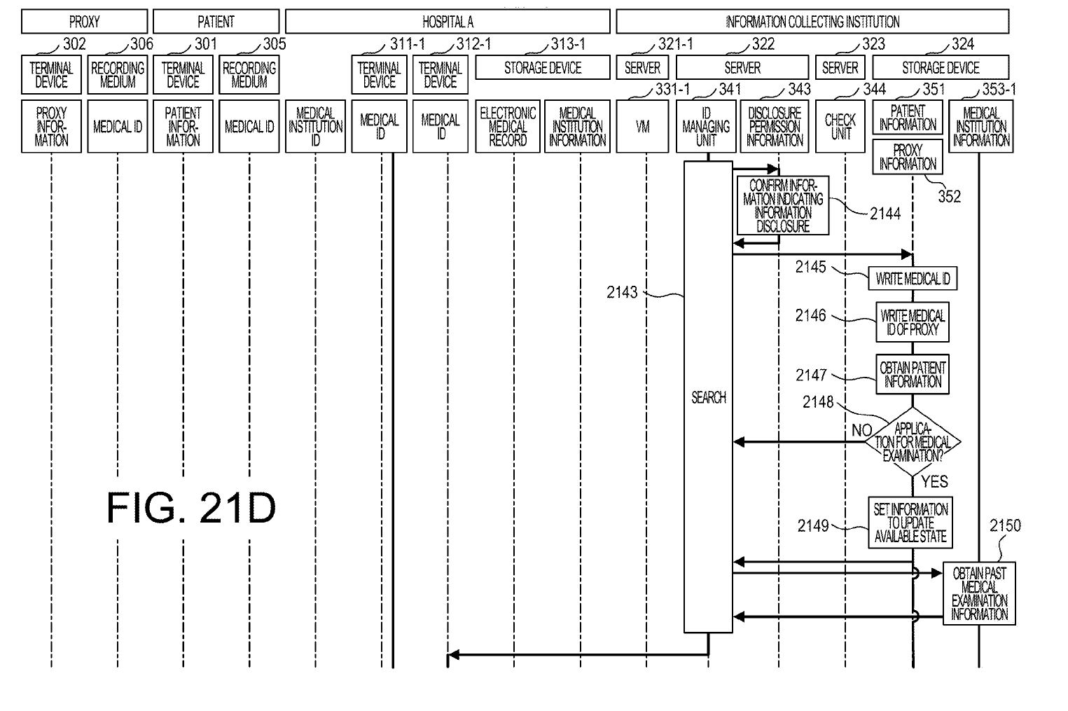

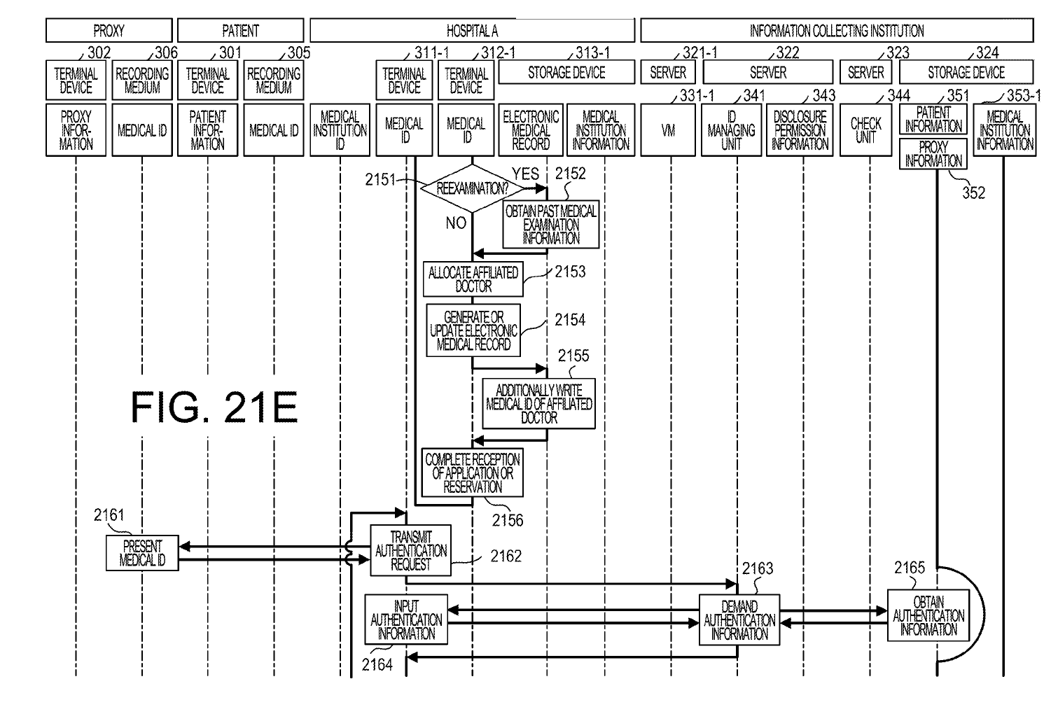

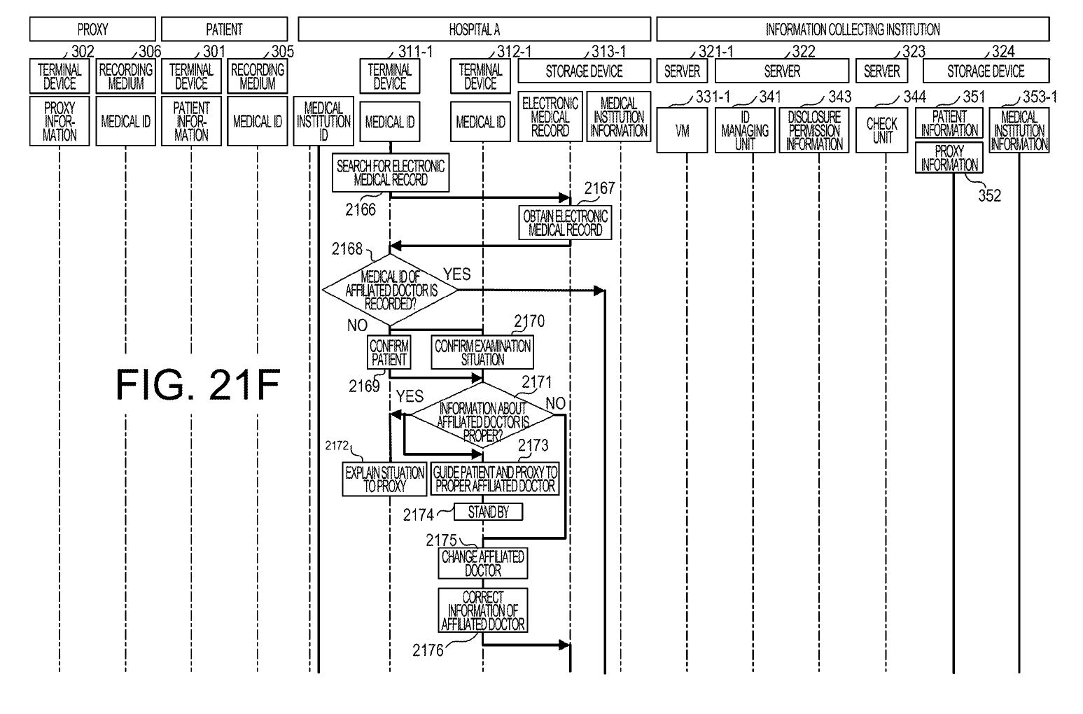

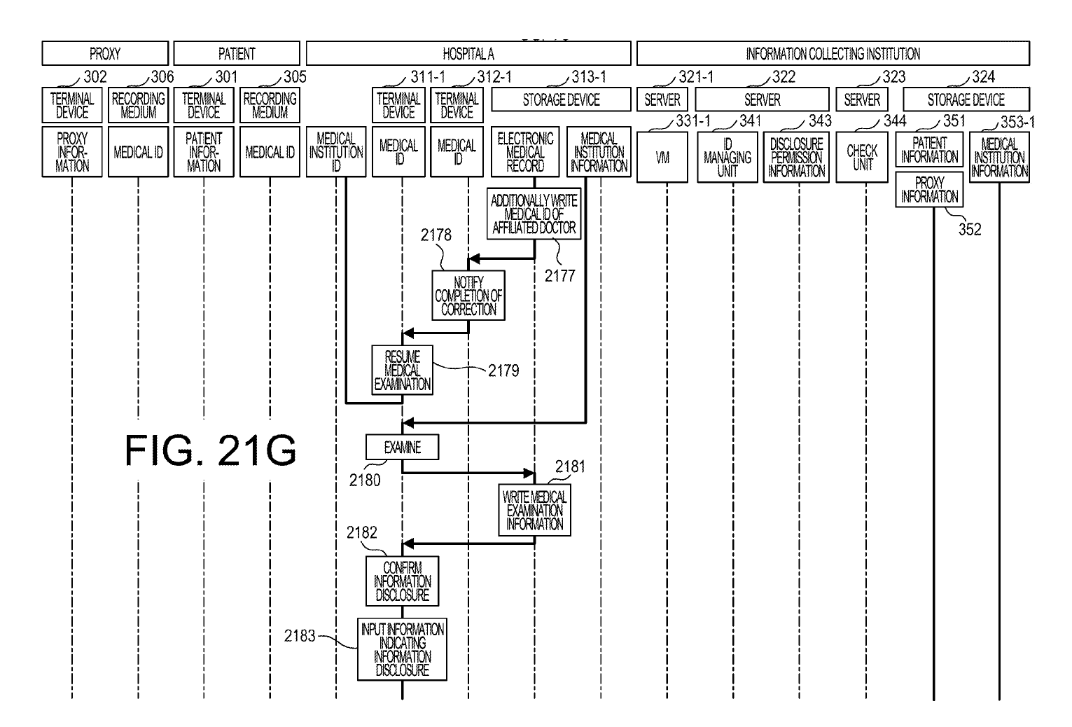

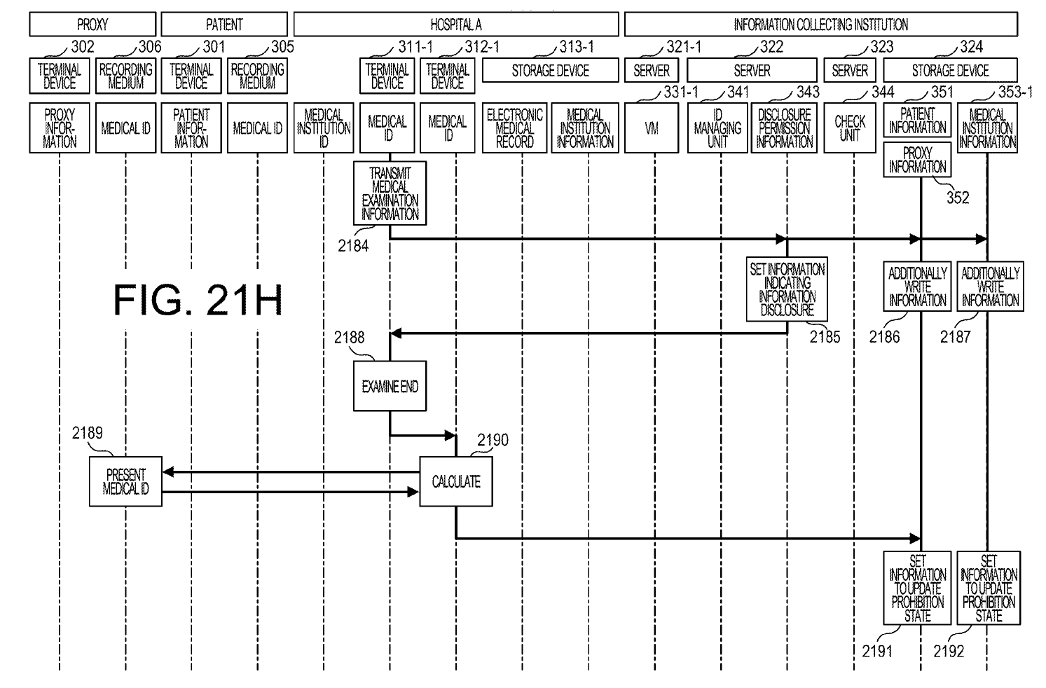

FIGS. 21A to 21H illustrate an example of a medical examination sequence when a patient is examined in the hospital A based on identity confirmation of a proxy when it is difficult to perform identity confirmation of the patient. Processing of steps 2111 to 2119 is the same as the processing of steps 1711 to 1719 of FIG. 17A.

Next, the proxy presents the recording medium 306 at the reception when the patient is examined in the hospital A or notifies the medical ID of the proxy through a phone and the like when making a reservation of a medical examination (step 2121). The office employee inputs the medical ID recorded in the recording medium 306 or the medical ID notified from the proxy to the terminal device 312-1 (step 2122).

Next, the office employee inquires whether the proxy possesses the recording medium 305 of the patient (step 2123). When the proxy possesses the recording medium 305 (step 2123, “YES”), the proxy presents the recording medium 305 (step 2124), and the office employee inputs the medical ID of the patient recorded in the recording medium 305 to the terminal device 312-1 (step 2125). Further, the terminal device 312-1 transmits an authentication request including the medical ID of the patient, the medical ID of the proxy, and the medical institution ID of the hospital A to the ID managing unit 341 (step 2126).

In the meantime, when the proxy does not possess the recording medium 305 (step 2123, “NO”), the terminal device 312-1 transmits an authentication request including the medical ID of the proxy and the medical institution ID of the hospital A to the ID managing unit 341 (step 2126).

Next, the ID managing unit 341 checks the proxy identically to step 1915 of FIG. 19A (step 2127). When the medical ID of the patient is included in the received authentication request, the proxy check processing that is the same as that illustrated in FIGS. 20A to 20C is performed.

In the meantime, when the medical ID of the patient is not included in the received authentication request, the ID managing unit 341 transmits a check request including only the medical ID of the proxy to the check unit 344 in step 2001 of FIG. 20A. Then, in step 2003, the check unit 344 obtains information corresponding to the medical ID of the proxy from the proxy information 352. In this case, the check unit 344 does not obtain the information corresponding to the medical ID of the patient from the patient information 351, so that processing of steps 2008, 2011, and 2015 is skipped.

As described above, eligibility of the proxy is checked during the reception of the application or the reservation of the medical examination, so that the application or the reservation acting on behalf of the patient by an ineligible proxy may be prevented.

The ID managing unit 341 determines whether the proxy is rejected based on a result of the proxy check processing (step 2128). When the proxy is rejected (step 2128, “YES”), the ID managing unit 341 notifies the terminal device 312-1 of the rejection of the authentication, and the office employee performs re-confirmation of the identity of the proxy (step 2133).

In the meantime, when the proxy is not rejected (step 2128, “NO”), the ID managing unit 341 requests the authentication information used in the identity authentication of the proxy from the terminal device 312-1 (step 2129). Then, the proxy inputs the authentication information to the terminal device 312-1 (step 2130), and the terminal device 312-1 transmits the input authentication information to the ID managing unit 341.

Next, the ID managing unit 341 obtains the authentication information corresponding to the medical ID of the proxy from the proxy information 352 of the storage device 324 (step 2131), and checks whether the authentication is successful (step 2132). For example, when the authentication information received from the terminal device 312-1 matches the authentication information obtained from the proxy information 352, the ID managing unit 341 determines that the authentication is successful.

When the authentication fails (step 2132, “NO”), the ID managing unit 341 notifies the terminal device 312-1 of the failure of the authentication, and the office employee performs re-confirmation of the identity of the proxy (step 2133).

In the meantime, when the authentication is successful (step 2132, “YES”), the ID managing unit 341 obtains the medical ID of the client corresponding to the medical ID of the proxy from the proxy information 352 (step 2134). Then, the ID managing unit 341 obtains basic patient information corresponding to the medical ID of the client obtained from the patient information 351 of the storage device 324 (step 2135).

Next, the ID managing unit 341 checks whether the medical ID of the patient is included in the received authentication request (step 2136). When the medical ID of the patient is included in the received authentication request (step 2136, “YES”), the ID managing unit 341 checks whether the medical ID of the patient matches the medical ID of the client obtained in step 2134 (step 2142). When the medical ID of the patient does not match the medical ID of the client (step 2142, “NO”), the ID managing unit 341 notifies the terminal device 312-1 of the failure of the authentication, and the office employee performs re-confirmation of the identity of the proxy (step 2133).

In the meantime, when the medical ID of the patient is not included in the authentication request (step 2136, “NO”), the ID managing unit 341 transmits the basic patient information of the client obtained in step 2135 to the terminal device 312-1 (step 2137).

The office employee inquires of the proxy basic information, such as a name, an address, gender, and a birth date, about the patient (step 2138), and compares the basic information answered by the proxy and the basic patient information received from the ID managing unit 341 (step 2139). When the office employee determines that the basic information answered by the proxy does not match the received basic patient information (step 2139, “NO”), the office employee considers that the patient is unidentified.

In the meantime, when the office employee determines that the basic information answered by the proxy matches the received basic patient information (step 2139, “YES”), the office employee inputs the determination to the terminal device 312-1 (step 2141). Then, the terminal device 312-1 transmits information indicating the input determination to the ID managing unit 341. Next, the ID managing unit 341 performs a check of step 2142 and determines that the medical ID of the patient matches the medical ID of the client based on the information received from the terminal device 312-1.

When the medical ID of the patient matches the medical ID of the client (step 2142, “YES”), the ID managing unit 341 searches for medical treatment information corresponding to the medical ID of the patient in the storage device 324 (step 2143). First, the ID managing unit 341 obtains information corresponding to the medical ID of the patient from the disclosure permission information 343, and checks the item of which the information disclosure is permitted (step 2144).

Next, the ID managing unit 341 writes the medical institution ID of the hospital in the item of the examining medical institution corresponding to the medical ID of the patient of the patient information 351 (step 2145) and writes the medical ID of the proxy (step 2146). Accordingly, it is recorded in the patient information 351 that the proxy acts for the application or the reservation. Then, the ID managing unit 341 obtains the basic patient information and the basic medical information corresponding to the medical ID of the patient from the patient information 351 (step 2147).

Next, the ID managing unit 341 checks whether a chance of the authentication request is an application for a medical examination (step 2148). When the chance of the authentication request is the application for the medical examination (step 2148, “YES”), the ID managing unit 341 sets the information about the patient included in the patient information 351 to an update available state (step 2149). In the meantime, when the chance of the authentication request is a reservation of the medical examination (step 2148, “NO”), the ID managing unit 341 performs subsequent processing to step 2150.

Next, the ID managing unit 341 obtains past medical examination information corresponding to the medical ID of the patient from the medical institution information 353-i (step 2150). Accordingly, the medical examination information of which the information disclosure is permitted among the plurality of elements of the medical examination information included in the medical institution information 353-1 of the hospital A and the medical institution information 353-2 of the hospital B is obtained. Then, the ID managing unit 341 notifies the terminal device 312-1 of the success of the authentication, and transmits the patient information 351 and the information obtained from the medical institution information 353-i to the terminal device 312-1.

Next, the terminal device 312-1 checks whether the current examination is a re-examination (step 2151). When the current examination is the re-examination (step 2151, “YES”), the terminal device 312-1 obtains the past medical examination information of the patient from the electronic medical record of the storage device 313-1 (step 2152). However, when the current examination is the first examination (step 2151, “NO”), the terminal device 312-1 performs processing subsequent to step 2153.

Next, the office employee allocates an affiliated doctor to the patient (step 2153), and the terminal device 312-1 generates or updates the electronic medical record of the patient (step 2154). Accordingly, the information received from the ID managing unit 341, the medical ID of the proxy, and the medical ID of the allocated affiliated doctor are additionally written in the electronic medical record (step 2155). Then, the terminal device 312-1 completes the reception of the application or the reservation of the patient (step 2156), and repeats processing subsequent to step 2122 to a next patient.

In the examination room, the proxy presents the recording medium 306 to the doctor (step 2161), the doctor inputs the medical ID recorded in the recording medium 306 and the medical ID of the doctor to the terminal device 311-1 (step 2162). Then, the terminal device 311-1 transmits an authentication request including the medical ID of the proxy to the ID managing unit 341.

Next, the ID managing unit 341 request authentication information used for identity authentication of the proxy from the terminal device 311-1 (step 2163). Then, the proxy inputs the authentication information to the terminal device 311-1 (step 2164), and the terminal device 311-1 transmits the input authentication information to the ID managing unit 341.

Next, the ID managing unit 341 obtains the authentication information corresponding to the medical ID of the proxy from the proxy information 352 of the storage device 324 (step 2165), and performs identity authentication of the proxy by using the obtained authentication information and the authentication information received from the terminal device 311-1.

Next, the terminal device 311-1 searches for the electronic medical record of the patient in which the medical ID of the proxy is recorded in the storage device 313-1 (step 2166), and obtains the electronic medical record (step 2167). Then, the terminal device 311-1 checks whether the medical ID of the doctor is recorded in the obtained electronic medical record (step 2168).

When the medical ID of the doctor is not recorded in the electronic medical record (step 2168, “NO”), the terminal device 311-1 requests the confirmation of an examination situation from the terminal device 312-1. The doctor confirms the patient (step 2169), and the office employee confirms the examination situation (step 2170). Then, the office employee checks whether the information of the affiliated doctor recorded in the electronic medical record is proper (step 2171).

When the information of the affiliated doctor is proper (step 2171, “YES”), there is a possibility that the proxy may make a mistake of an incorrect examination room. Accordingly, the doctor explains the situation to the proxy (step 2172), and the office employee guides the patient and the proxy to an examination room of the proper affiliated doctor (step 2173), and the patient and the proxy stand by in the guided examination room (step 2174).

In the meantime, when the information of the affiliated doctor is not proper (step 2171, “NO”), the office employee changes an affiliated doctor (step 2175), and the terminal device 312-1 corrects the information about the affiliated doctor recorded in the electronic medical record (step 2176). Accordingly, a medical ID of the corrected affiliated doctor is additionally written in the electronic medical record (step 2177).

Next, the terminal device 312-1 notifies the terminal device 311-1 of the completion of the correction (step 2178), and the doctor resumes an examination of the patient (step 2179), and the terminal device 311-1 repeats processing subsequent to step 2162.

When the medical ID of the doctor is recorded in the electronic medical record (step 2168, “YES”), the doctor examines the patient, and inputs medical examination information indicating a result of the examination to the terminal device 311-1 (step 2180). Then, the terminal device 311-1 writes the medical examination information in the electronic medical record of the storage device 313-1 (step 2181).

Next, the doctor confirms whether to permit the disclosure of the medical examination information of the patient to the proxy (step 2182), and inputs the disclosure or non-disclosure of the information to the terminal device 311-1 based on an answer of the proxy (step 2183). Then, the terminal device 311-1 transmits the medical ID of the patient, the medical ID of the doctor, the medical examination information, and the information indicating the disclosure/non-disclosure of the information to the ID managing unit 341 (step 2184).

The ID managing unit 341 sets the disclosure or non-disclosure of the information in the disclosure permission information 343 based on the information received from the terminal device 311-1 (step 2185). Next, the ID managing unit 341 additionally writes an examination date in the item of the examining medical institution corresponding to the medical ID of the patient of the patient information 351 (step 2186). Further, the ID managing unit 341 additionally writes the examination date, the examination department, the affiliated doctor, and the medical examination information in the information about the patient corresponding to the medical ID of the patient of the medical institution information 353-1 (step 2187).

The doctor terminates the examination (step 2188), the proxy presents the recording medium 306 at the reception (step 2189), and the office employee inputs the medical ID recorded in the recording medium 306 to the terminal device 312-1 and performs a calculation (step 2190). Then, the terminal device 312-1 notifies the ID managing unit 341 of the termination of the medical examination.

Next, the ID managing unit 341 sets the information about the patient included in the patient information 351 to the update prohibition state (step 2191), and sets the information about the hospital A included in the medical institution information 353-1 to the update prohibition state (step 2192).

In the information processing system of FIG. 3, the information providing institution and the information using institution may be other institutions in addition to the hospital which provides and uses medical examination information of a patient. For example, an example of the information providing institution and the information using institution may include a store providing and using purchasing information of customers, an educational institution such as a school and a preparatory school, providing and using grade information of students, or a financial institution, such as a bank, providing and using bank balances, trade performance, and the like of customers.

When the information providing institution and the information using institution are stores, purchasing information of a customer is collected as personal information, and is provided to another store on the premise of permission by a customer or a proxy. When the information providing institution and the information using institution are educational institutions, grade information of a student is collected as personal information, and is provided to another educational institution on the premise of permission by a student or a proxy. When the information providing institution is a financial institution, a bank balance, trade performance of a customer, and the like are collected as personal information, and is provided to another financial institution on the premise of permission by a customer or a proxy.

The configuration of the information processing apparatus 101 of FIG. 1 is simply an example, and some constituent elements may be omitted or changed according to a usage or a condition of the information processing apparatus 101. For example, the memory unit 102 may also be provided inside the information processing apparatus 101.

The configuration of the information processing system of FIG. 3 is simply an example, and some constituent elements may be omitted or changed according to a usage or a condition of the information processing system. For example, when the VM 331-1 is operable within the server 321-1, the server 321-2 may be omitted. When the check unit 344 is provided within the server 322, the server 323 may be omitted.

The operation sequences of the flowchart of FIG. 2, FIGS. 16A and 16B to FIGS. 21A to 21H are simply examples, and some processing may be omitted or changed according to a configuration or a condition of the information processing system. For example, in the proxy check processing of FIGS. 20A to 20C, the check unit 344 need not to perform all of the checks of steps 2004 to 2011, step 2014, and step 2015, and may also perform only some of the checks. An operation sequence in the case where the information processing system includes three or more hospital systems is the same as the operation sequences of FIGS. 16A and 16B to FIGS. 21A to 21H.

The patient information 351 and the proxy information 352 of FIGS. 4 to 9 and the medical institution information 353-i of FIGS. 10A and 10B to FIG. 15 are simply examples, and some or the entirety of the items may be omitted or changed according to a usage or a condition of the information processing system.

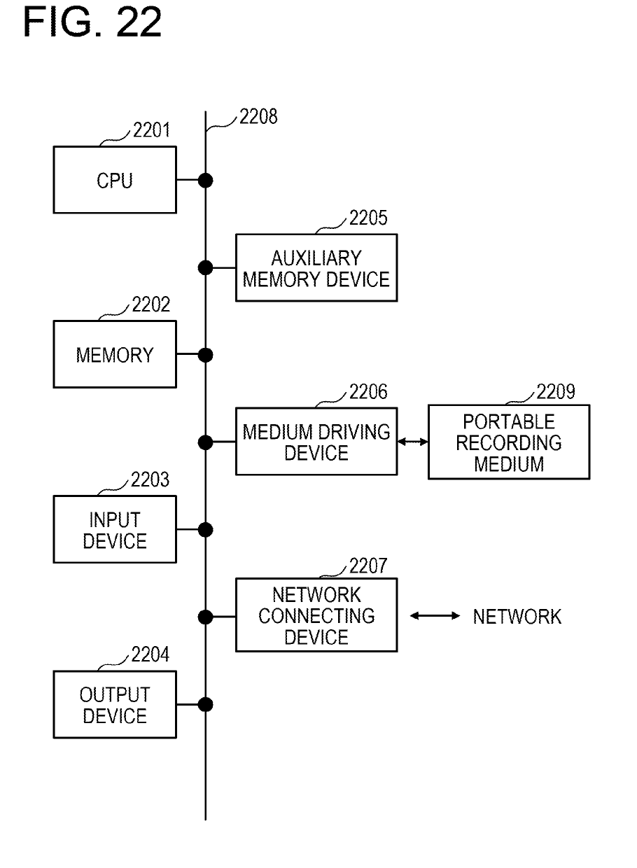

FIG. 22 illustrates a configuration example of hardware of the information processing apparatus 101 of FIG. 1 and the information processing apparatus used as the server 322 of FIG. 3. The information processing apparatus of FIG. 22 includes a central processing unit (CPU) 2201, a memory 2202, an input device 2203, an output device 2204, an auxiliary memory device 2205, a medium driving device 2206, and a network connection device 2207. The constituent elements are connected with one another by a bus 2208.

The memory 2202 may be a semiconductor memory such as, for example, a read only memory (ROM), a random access memory (RAM), and a flash memory, and stores a program and data used for processing. The memory 2202 may be used as the memory unit 342 of FIG. 3.

The CPU 2201 (processor) executes a program by using, for example, the memory 2202, thereby being operated as the obtaining unit 111 of FIG. 2 and the ID managing unit 341 of FIG. 3.

The input device 2203 is, for example, a keyboard and a pointing device, and is used for an input of an instruction or information from an operator or a user. The output device 2204 is, for example, a display device, a printer, and a speaker, and is used for an output of a result of an inquiry or a processing result to an operator or a user.

The auxiliary memory device 2205 is, for example, a magnetic disk device, an optical disk device, a magneto-optical disk device, and a tape device. The auxiliary memory device 2205 may be a hard disk drive. The information processing apparatus may store a program and data in the auxiliary memory device 2205, and may load the program and the data in the memory 2202 and use the program and the data. The auxiliary memory device 2205 may be used as the memory unit 342 of FIG. 3.

The medium driving device 2206 drives a portable recording medium 2209, and accesses the recorded contents. The portable recording medium 2209 is a memory device, a flexible disk, an optical disk, and a magneto-optical disk. The portable recording medium 2209 may be a DVD, a compact disk read only memory (CD-ROM), a universal serial bus (USB) memory, and the like. An operator or a user may store a program and a data in the portable recording medium 2209, and may load the program and the data in the memory 2202 and use the program and the data.

As described above, the computer readable recording medium storing a program and data used for processing is a physical (non-temporary) recording medium, such as the memory 220, the auxiliary memory device 2205, or the portable recording medium 2209.

The network connection device 2207 is a communication interface which is connected to a communication network, such as a local area network (LAN) and a wide area network (WAN), and performs data conversion accompanying the communication. The information processing apparatus receives a program and data from an external device via the network connection device 2207, and may load the program and the data in the memory 2202 and use the program and the data.

The network connection device 2207 may be used as the output unit 112 of FIG. 1. When the information processing apparatus is the server 322 of FIG. 3, the network connection device 2207 is operated as the output unit 112, and transmits medical examination information of a patient which is obtained by the ID managing unit 341 from the storage device 324 to the hospital system 303-i.

The information processing apparatus does not need to include all of the constituent elements of FIG. 22, and some constituent elements may be omitted according to a usage or a condition of the information processing apparatus. For example, when it is not necessary to input an instruction or information from an operator or a user, the input device 2203 may be omitted, and when it is not necessary to output a result of an inquiry or processing to an operator or a user, the output device 2204 may be omitted. When the portable recording medium 2209 is not used, the medium driving device 2206 may be omitted.

The same information processing apparatus as that of FIG. 22 may be used as the terminal device 301, the terminal device 302, the terminal device 311-i, the terminal device 312-i, the storage device 313-i, the server 321-i, the server 323, and the storage device 324 of FIG. 3. In this case, the CPU 2201 is operated as the check unit 344 by executing a program by using the memory 2202. Further, the CPU 2201 operates the VM 331-i by executing a program by using the memory 2202.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to an illustrating of the superiority and inferiority of the invention. Although the embodiments of the present invention have been described in detail, it should be understood that the various changes, substitutions, and alterations could be made hereto without departing from the spirit and scope of the invention.

What is claimed is:

1. An information processing apparatus, comprising:
a memory; and
a processor coupled to the memory and the processor configured to:
store personal identification information indicating a person and proxy identification information indicating a proxy who acts on behalf of the person by associating the personal identification information and the proxy identification information with each other;
obtain the personal identification information from the memory, which stores the personal identification information and personal information of the person in association with each other, based on the proxy identification information presented by the proxy;
obtain the personal information from the memory based on the personal identification information obtained from the memory; and
output the personal information obtained.

2. The information processing apparatus according to claim 1, wherein the personal information includes information about the person provided from a different information providing institution from an information using institution which uses the personal information,
the processor is configured to receive the proxy identification information presented by the proxy to the information using institution from the information using institution, and obtain the personal identification information from the memory based on the received proxy identification information, and
the processor is configured to transmit the personal information to the information using institution.

3. The information processing apparatus according to claim 1, wherein the processor is configured to transmit a proxy check request including the proxy identification information to a check unit, and when the processor receives a check result indicating that the proxy is eligible from the check unit, the processor obtains the personal identification information from the memory based on the proxy identification information, and when the processor receives a check result indicating that the proxy is rejected, the processor does not obtain the personal identification information from the memory unit.

4. The information processing apparatus according to claim 3, wherein when the proxy indicated by the proxy identification information dies, when the proxy indicated by the proxy identification information is registered in a list of people who are undesirable as a proxy, when the number of people who the proxy indicated by the proxy identification information acts on behalf of is larger than a predetermined number, or when an address of the proxy indicated by the proxy identification information is not within a predetermined range based on an address of the person, the check result indicates that the proxy is rejected.

5. A non-transitory computer-readable recording medium having stored therein a program for causing a computer to execute a processing, the processing including:
storing personal identification information indicating a person and proxy identification information indicating a proxy who acts on behalf of the person by associating the personal identification information and the proxy identification information with each other, and obtaining the personal identification information from a memory, which stores the personal identification information and personal information of the person in association with each other, based on the proxy identification information presented by the proxy; and
obtaining the personal information from the memory based on the obtained personal identification information, and
outputting the obtained personal information.

6. The non-transitory computer-readable recording medium according to claim 5, wherein the personal information includes information about the person provided from a different information providing institution from an information using institution which uses the personal information, and
the computer receives the proxy identification information presented by the proxy to the information using institution from the information using institution, obtains the personal identification information from the memory based on the received proxy identification information, and transmits the personal information to the information using institution.

7. The non-transitory computer-readable recording medium according to claim 5, wherein the computer transmits a proxy check request including the proxy identification information to a check unit, and when the computer receives a check result indicating that the proxy is eligible from the check unit, the computer obtains the personal identification information from the memory based on the proxy identification information, and when the computer receives a check result indicating that the proxy is rejected from the check unit, the computer does not obtain the personal identification information from the memory.

8. The non-transitory computer-readable recording medium according to claim 7, wherein when the proxy indicated by the proxy identification information dies, when the proxy indicated by the proxy identification information is registered in a list of people who are undesirable as a proxy, when the number of people who the proxy indicated by the proxy identification information acts on behalf of is larger than a predetermined number, or when an address of the proxy indicated by the proxy identification information is not within a predetermined range based on an address of the person, the check result indicates that the proxy is rejected.

9. A method of processing information, the method comprising:
storing, by a computer, personal identification information indicating a person and proxy identification information indicating a proxy who acts on behalf of the person by associating the personal identification information and the proxy identification information with each other, and obtaining the personal identification information from a memory, which stores the personal identification information and personal information of the person in association with each other, based on the proxy identification information presented by the proxy, and
obtaining the personal information from the memory unit based on the obtained personal identification information, and
outputting the obtained personal information.

10. The method according to claim 9, wherein the personal information includes information about the person provided from a different information providing institution from an information using institution which uses the personal information,
the computer receives the proxy identification information presented by the proxy to the information using institution from the information using institution, obtains the personal identification information from the memory based on the received proxy identification information, and transmits the personal information to the information using institution.

11. The method according to claim 9, wherein the computer transmits a proxy check request including the proxy identification information to a check unit, and when the computer receives a check result indicating that the proxy is eligible from the check unit, the computer obtains the personal identification information from the memory based on the proxy identification information, and when the computer receives a check result indicating that the proxy is rejected from the check unit, the computer does not obtain the personal identification information from the memory.

12. The method according to claim 11, wherein when the proxy indicated by the proxy identification information dies, when the proxy indicated by the proxy identification information is registered in a list of people who are undesirable as a proxy, when the number of people who the proxy indicated by the proxy identification information acts on behalf of is larger than a predetermined number, or when an address of the proxy indicated by the proxy identification information is not within a predetermined range based on an address of the person, the check result indicates that the proxy is rejected.
